(19) United States
(12) Patent Application Publication
(10) Pub. No.: US 2026/0026927 A1
(43) Pub. Date: Jan. 29, 2026

(54) MINIMALLY INVASIVE IMPLANT PROCEDURE AND SYSTEM

(71) Applicant: ESTABLISHMENT LABS S.A., ALAJUELA (CR)

(72) Inventors: Juan José Chacón Quirós, Alajuela (CR); Manuel Chacón Quirós, Alajuela (CR); Charles Ranquist, Alajuela (CR); Marcos Sforza, Alajuela (CR); Roberto De Mezerville, Alajuela (CR); Johan Andersson, Alajuela (CR); Thomas Fuller, Alajuela (CR); Jeffry Fassero Taglioli, Alajuela (CR); Mario Ramirez, Alajuela (CR)

(21) Appl. No.: 19/144,197

(22) PCT Filed: Dec. 28, 2023
(86) PCT No.: PCT/IB2023/000786

§ 371 Date: Jun. 27, 2025

Related U.S. Application Data
(60) Provisional application No. 63/435,929, filed on Dec. 29, 2022.

(51) Int. Cl. A61F 2/12; A61B 17/02; A61B 17/34; A61B 90/00; A61M 25/10

(52) U.S. Cl. CPC A61F 2/12; A61B 17/3468; A61B 90/02; A61B 2017/0225; A61M 25/1002; A61M 25/10183

(57) ABSTRACT

An implant insertion system including a tissue expander or separator including an inflatable balloon with a curved posterior side when inflated and an implant having a curved posterior side when positioned against an anatomical surface, wherein the curved posterior side of the implant substantially matches the curved posterior side of the tissue expander. The implant can have an anterior side substantially similar to the posterior side. The implant insertion system can have an injector configured to insert the implant into a target location.

### CLAIM OF PRIORITY

This application claims the benefit of U.S. Provisional Patent Application Ser. No. 63/435,929, filed on Dec. 29, 2022, the benefit of priority of which is claimed hereby, and which is incorporated by reference herein in its entirety.

### TECHNICAL FIELD

The present disclosure relates generally to implants, tools, systems, and surgical methods for insertion of implants into a human body. More specifically, the present disclosure relates to compressible breast implants, breast implantation tools, and surgical methods for performing minimally invasive breast implantation procedures.

### BACKGROUND

Breast implants are among the largest implantable medical devices to be inserted in the human body. Insertion of a breast implant into a human body usually requires a large incision to be made in the patient's body proximate to the target site for the implant. The large incision is usually required not only for passage of the breast implant into the patient's body, but also to allow room for the tools required to perform the procedure. It can be appreciated that large incisions can increase the likelihood of tissue damage, inflammatory responses, and the like.

Breast implantation surgical procedures can be time-intensive, invasive procedures with long recovery times. Due to the length of time of the surgical procedure, the incisions, and the manipulations a medical professional must perform during the surgical procedure, the patient is usually subjected to general anesthesia, so the patient is not conscious during the surgical procedure. After the surgical procedure is completed, the patient can have large, visible scars from where the medical professional made incisions to properly place the implant in the patient's body. The patient can also face complications from having large incisions made in the patient's body to facilitate proper insertion of the implant. A patient can also have an uncomfortable or painful recovery process as the implant is accepted in the patient's body and as the incisions heal.

### SUMMARY

The present inventors have recognized, among other things, that minimally invasive devices and methods of use can aid in solving the problems associated with surgical implantation procedures which require large incisions to be made in a patient's body.

The present inventors have recognized that implants which are substantially uniform in shape in at least one orientation can aid in solving the problem of inserting the implant in the correct orientation. The present inventors have also recognized that inserting implants having rounded posterior and anterior surfaces into cavities that are prepared with minimal damage to the connective tissue such as the ligaments or soft tissue can result in a more natural anatomical appearance. Additionally, the present inventors have recognized that performing minimally invasive breast implantation surgical procedures can reduce the length of the surgical procedure, minimize damage to the patient's body tissue, avoid the use of general anesthesia, and promote a quicker and less painful recovery process. An example implant insertion system can include a tissue expander, or tissue separator, with an inflatable balloon, the balloon having a curved posterior side when inflated and an implant having a curved posterior side when positioned against an anatomical surface, wherein the curved posterior side of the implant substantially matches the curved posterior side of the tissue expander.

For example, an implant insertion system that is compatible with an implant with having, for example, an anterior side and a posterior side that are curved in a substantially similar manner and the longitudinal axis is positioned substantially equidistant from the anterior apex and the posterior apex can be beneficial. The implant system can also have additional components such as a tissue expanding or separating balloon that can be sized and have a similar profile to the desired implant.

An implant insertion system is contemplated that includes, for example, an injector including a nozzle that defines a housing and an implant. The injector of the implant insertion system can include a handle detachably coupled with the nozzle, where the handle includes an expandable membrane configured to expand into the nozzle with a supply of a pressurized fluid. The implant can be configured so the anterior side and posterior side of each projects a substantially similar distance away from the longitudinal axis in the first configuration. The implant can also be configured so the anterior side and the posterior side are substantially uniformly compressed in the second configuration. The implant can be a breast implant.

The present inventors have recognized a method of inserting and implanting an implant that can include using the balloon, for example, to form a nest-like cavity within a breast area of a patient's body that can assist in the implantation process. The example method can include making an incision, such as a small incision, and inserting a tissue expander separator such as a balloon into a target site of the patient's body the inflating the balloon at the target site between at least two layers of connective tissue to separate the at least two layers of connective tissue and form a cavity with a profile substantially matching a profile of a breast implant.

The method of inserting an implant can further include inserting the implant into the target site using an injector having a nozzle with a cavity, a nozzle end and a nozzle end opening, where the implant is compressed within the cavity of the nozzle between the nozzle end and an expandable membrane. The method can further include applying a force to the implant with an expandable membrane within the housing to expel the implant through the nozzle end and out of the nozzle end opening and into the target site.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various examples discussed in the present document.

FIG. 1A is a top view of an implant having an elliptical or spheroid shape in accordance with at least one example of the present disclosure.

FIG. 1B is a top view of an implant having an elliptical or spheroid shape in accordance with at least one example of the present disclosure.

FIG. 1C is a top view of an implant having an elliptical or spheroid shape with a substantially flat posterior side in accordance with at least one example of the present disclosure.

FIG. 1D is a side view of an implant having a tear drop shape in accordance with at least one example of the present disclosure.

FIGS. 1E-1F are charts illustrating the size, volume and protrusion distance in accordance with at least one example of the present disclosure.

FIG. 2 is a cross section of an implant in accordance with at least one example of the present disclosure.

FIGS. 3A-3D are cross sections of the shell of a breast implant in accordance with at least one example of the present disclosure.

FIG. 4 is a top view of a marking tool in accordance with at least one example of the present disclosure.

FIG. 5 is an isometric view of a cannula in accordance with at least one example of the present disclosure.

FIG. 6 is a side view of a tissue dissector in accordance with at least one example of the present disclosure.

FIG. 7A is an assembled, top view of a tissue expander in accordance with at least one example of the present disclosure.

FIG. 7B is an isometric view of a supporting extension of a tissue expander in accordance with at least one example of the present disclosure.

FIG. 8 is an exploded, isometric view of an injector in accordance with at least one example of the present disclosure.

FIG. 9 is a cross section of an injector in accordance with at least one example of the present disclosure.

FIG. 10 is a rear view of an injector in accordance with at least one example of the present disclosure.

FIG. 11 is a side view of an injector in accordance with at least one example of the present disclosure.

FIGS. 12A-12E are top views of nozzle ends and nozzle end openings in accordance with at least one example of the present disclosure.

FIG. 13 is a front view of a patient's torso in accordance with at least one example of the present disclosure.

FIGS. 14A-14D are illustrations of steps in a surgical procedure utilizing a marking tool in accordance with at least one example of the present disclosure.

FIG. 15 is an image of the areas for infiltration in accordance with at least one example of the present disclosure.

FIGS. 16A-16B are exemplary images of areas for use of a tissue dissector in accordance with at least one example of the present disclosure.

FIGS. 17A-17G are exemplary images of a procedure for using a tissue expander in accordance with at least one example of the present disclosure.

FIGS. 18A-18C are exemplary side profile images of locations of a balloon within a breast in accordance with at least one example of the present disclosure.

FIGS. 19A-19N are exemplary images of a procedure for inserting an implant into a breast with an injector in accordance with at least one example of the present disclosure.

FIG. 20A illustrates a cross sectional view of a breast in a reclined orientation with an implant inserted in accordance with at least one example of the present disclosure.

FIG. 20B illustrates a cross sectional view of a breast in an upright orientation with an implant inserted in accordance with at least one example of the present disclosure.

### DETAILED DESCRIPTION

The present disclosure relates to minimally invasive surgical procedures, devices and systems for inserting an implant into a target site in a patient's body. The present disclosure relates to implants inserted into a patient's body during, for example, a minimally invasive surgical procedure. The present disclosure also relates to devices, implements or tools which can be used in a minimally invasive surgical procedure for inserting an implant into a target site in a patient's body.

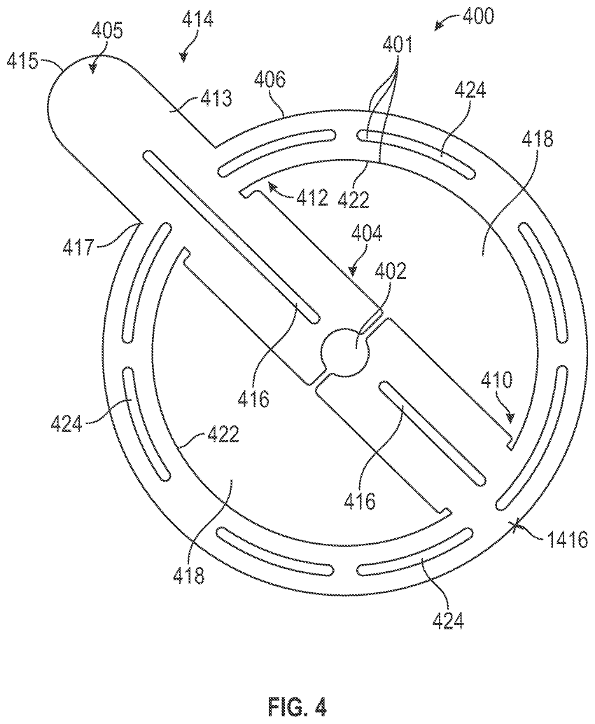

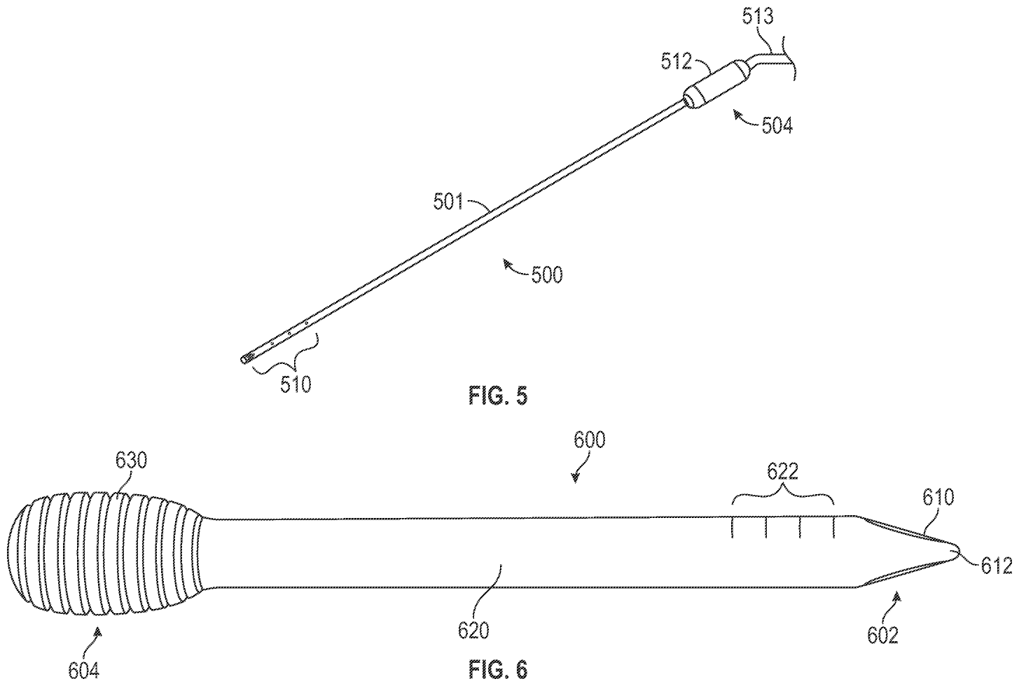

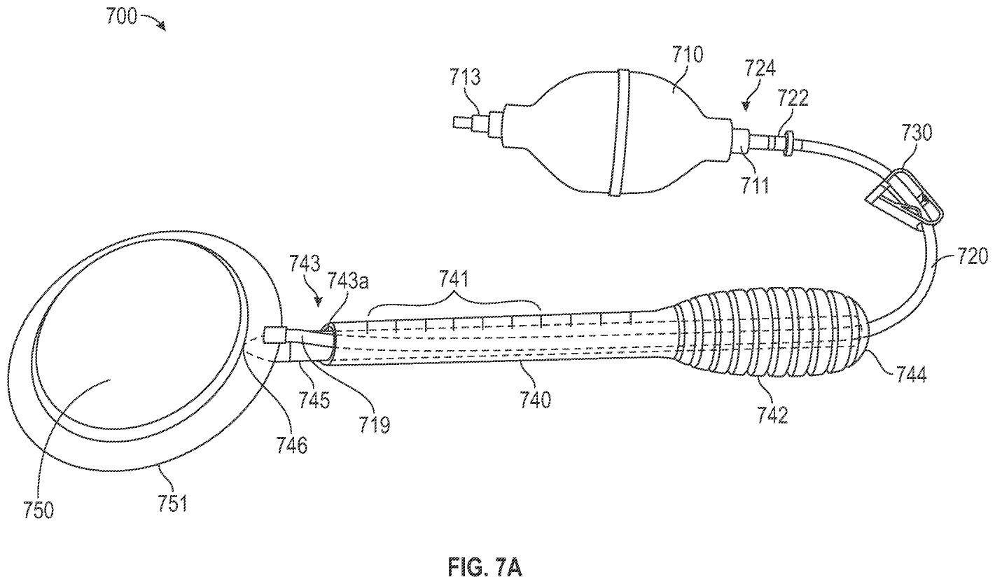

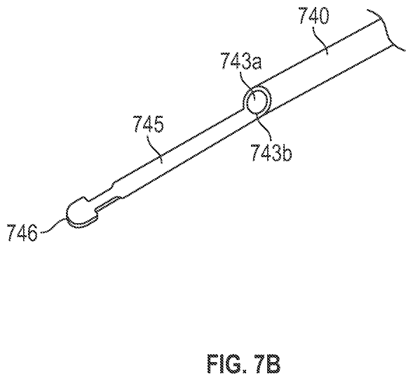

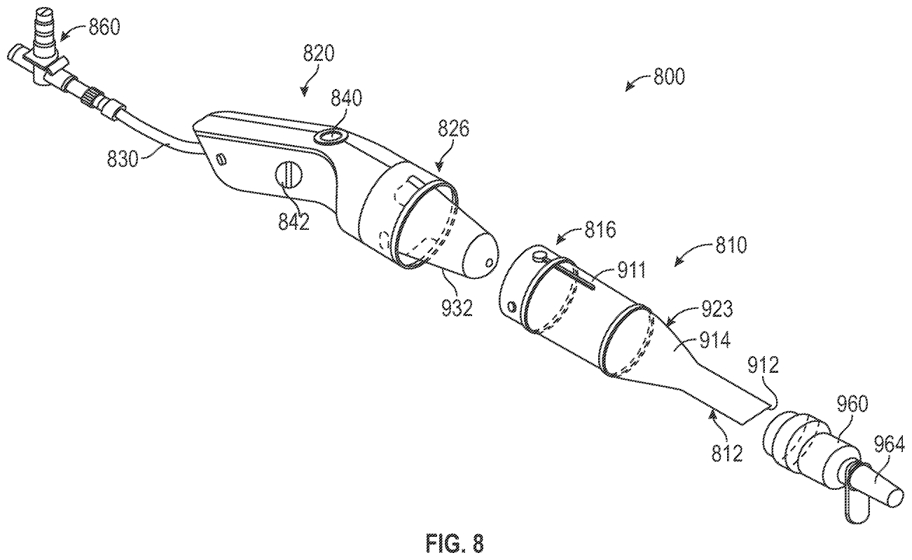

In an example surgical procedure, a medical professional, such as a doctor, a nurse, or a surgeon, can perform a surgical procedure to implant a breast implant in a target site. The implant surgical procedure can be a minimally invasive surgical procedure which can be performed as an in-patient or out-patient surgical procedure using local anesthesia. A minimally invasive surgical procedure for inserting an implant can utilize, for example, at least one, a few of, or all of the following tools: a marking tool 400 (as illustrated in FIG. 4), a cannula 500 (as illustrated in FIG. 5), a tissue dissector 600 (as illustrated in FIG. 6), a tissue expander or tissue separator (hereinafter “tissue expander” can refer to an implant such as a balloon that can separate layers of tissue) 700 (as illustrated in FIG. 7A), an injector 800 (as illustrated in FIG. 8) and an implant (such as implants 100, 150, 170, 180, 200 or 1110). Each of these tools, alone or together, can be used during a surgical procedure to insert and place the implant into a target site, such as a cavity, in a patient's body in a manner which the patient can be minimally impacted during the surgical procedure.

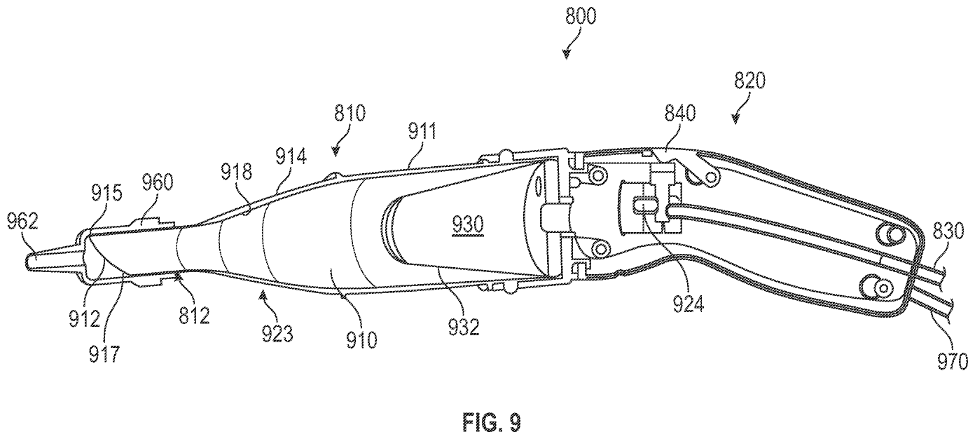

In an example surgical procedure, the surgeon can use the marking tool to indicate or guide where the target site for the implant is located, where an insertion incision can be made, how far the tissue dissector can be inserted, how much the balloon can be inflated and the approximate placement of the implant. After marking the breast accordingly, a medical professional can use the cannula to inject a local anesthetic into the breast area. Using a local anesthetic, the patient will, for example, be numb to the surgical procedure, yet remain conscious while undergoing the surgical procedure thereby offering a possibility for a shorter recovery. The medical professional can then make an incision at a desired location and gain access to the target site through the use of the tissue dissector or tissue separator. The tissue dissector can be inserted through the incision in such a way to form a channel or passage from the incision to the target site. The tissue expander can then be inserted through the small incision and traverse the passage until reaching the target site. The tissue expander can then be expanded within the target site to create a cavity sized and shaped similar to the implant without damaging a significant amount of connective tissue around the target site. The tissue expander can be expanded to a size and shape substantially similar to the implant, or expanded slightly beyond the size of the implant, to aid in hemostasis during a surgical procedure. After the tissue expander has, for example, been deployed for a specific period of time it can be deflated and subsequently removed through the small incision. In an example, the injector can be pre-loaded with a compressible implant. The injector can have a nozzle end (such as nozzle end 812 in FIG. 8) which can be designed to fit within the small incision and through the passage. The nozzle end can have a nozzle end opening (such as nozzle end opening 912 in FIG. 8) through which the implant can be expelled or ejected from the injector. To insert the implant into the target site, the nozzle end of the injector can be placed within the incision and the nozzle end can then extend within the passage. When the nozzle end of the injector is within the passage at a predetermined depth, the medical professional can activate an actuator (such as actuator 840 in FIG. 8) which can cause an expandable membrane (such as expandable membrane 932 in FIG. 9), or diaphragm, coupled to the handle (such as handle 820 in FIG. 8) of the injector, to apply a predetermined amount of pressure to the implant thereby expelling, or ejecting, the implant from the injector through the nozzle end opening. In this example, the implant can thereby be placed or implanted in the desired location of the target site. The medical professional can then remove the injector from the incision and close the incision. This process can then be repeated on an opposing side of the patient's body.

### Implant

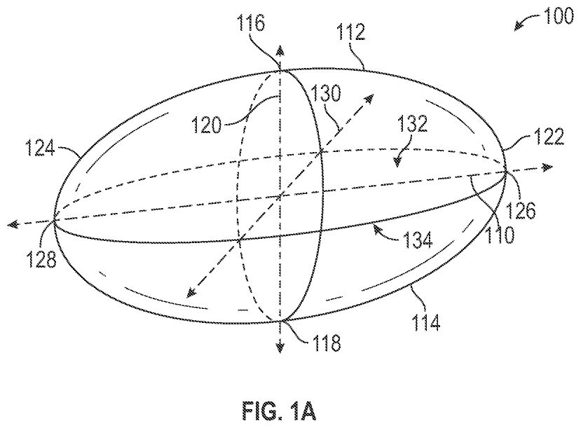

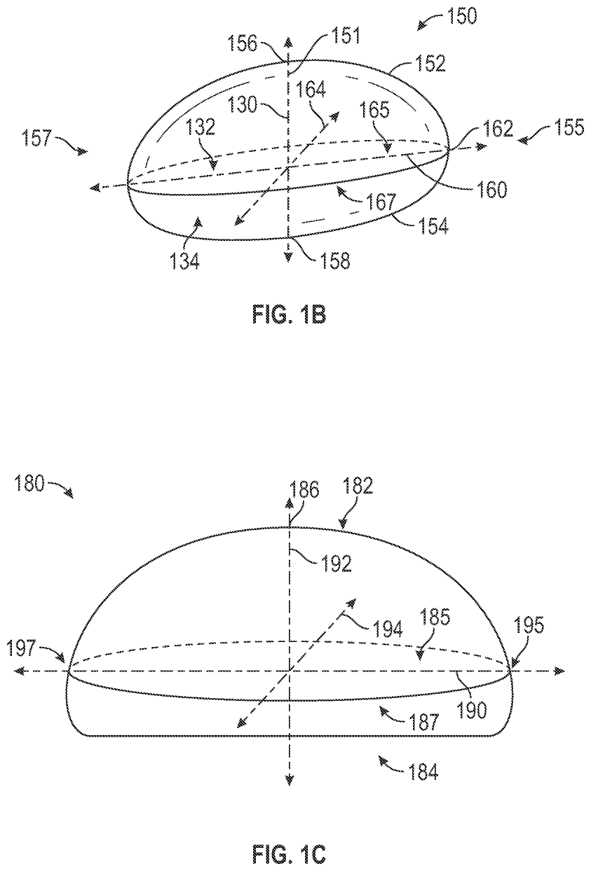

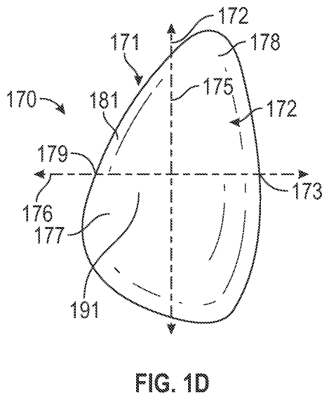

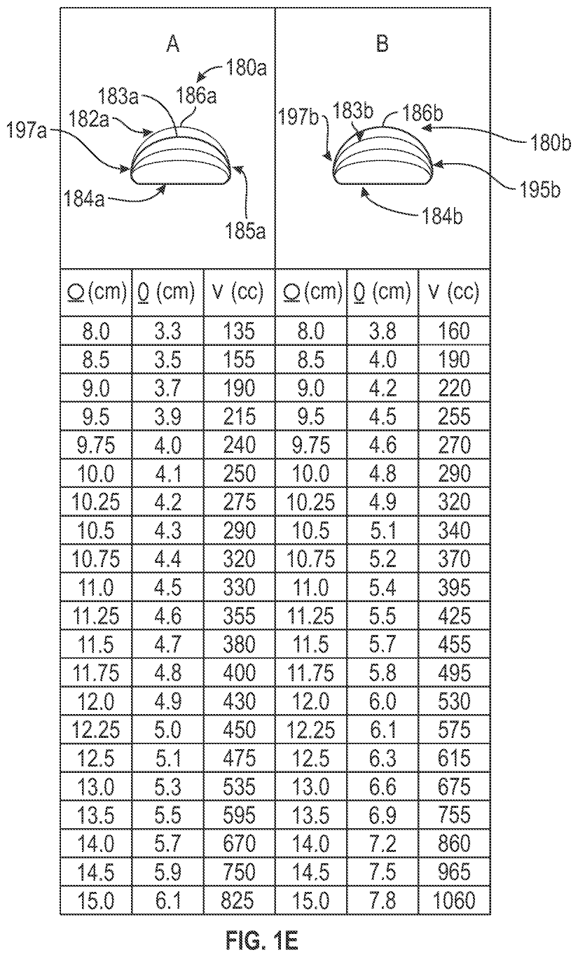

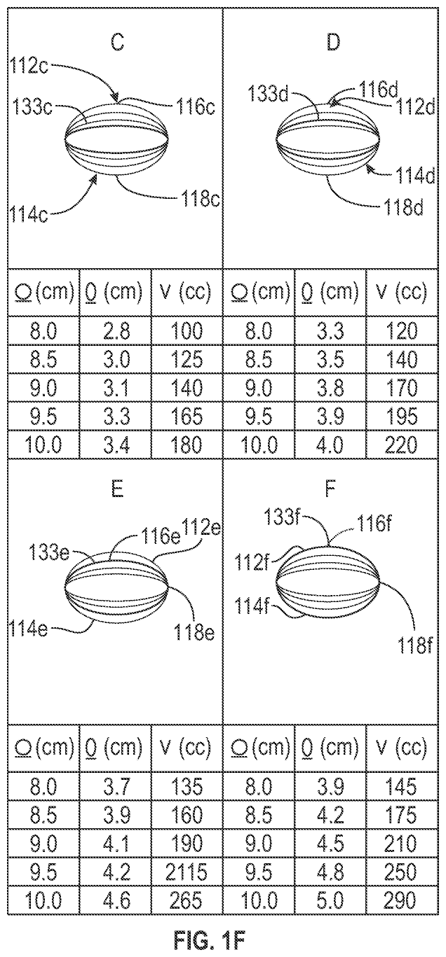

FIG. 1A illustrates a top view of an implant 100 of an implant insertion system having an elliptical or spheroid shape in accordance with at least one example of the present disclosure. The implant 100 can be substantially symmetrical about one or more axes, such as a longitudinal axis 110, a lateral axis 120 or a vertical axis 130.

The longitudinal axis 110 can separate the anterior side 112 from the posterior side 114. The longitudinal axis 110 can also refer to a longitudinal plane which traverses the implant 100, separating the anterior side 112 from the posterior side 114. The anterior side 112 can refer to the front of a patient or the orientation facing away from a medial orientation. For example, the side of a breast implant closest to the skin can be considered the anterior side 112. In another example, the side of a gluteal implant closest to the skin can be considered the anterior side 112. The posterior side 114 can refer to the side of the implant 100 closest to the back of a patient. For example, the side of a breast implant closest to the patient's ribs can be considered the posterior side 114. In another example, the side of a gluteal implant closest to the patient's pelvic region can be considered the posterior side 114.

As illustrated in FIG. 1A, the implant 100 can also be symmetrical about a lateral axis 120. The lateral axis 120 can separate a right side 122 of the implant 100 from the left side 124 of the implant 100. The lateral axis 120 can also refer to or represent a lateral plane which traverses the implant 100, separating the right side 122 of the implant 100 from the left side 124 of the implant 100. In various examples, the lateral axis 120 can be perpendicular to the longitudinal axis 110, the lateral axis 120 can be substantially perpendicular to the longitudinal axis 110, or the lateral axis 120 can be askew relative to the longitudinal axis 110.

As illustrated in FIG. 1A, the implant 100 can also be symmetrical about the vertical axis 130. The vertical axis 130 can separate the top, or superior, 132 portion of the implant 100 from the bottom, or inferior, 134 portion of the implant 100. The vertical axis 130 can also refer to or represent a vertical plane which traverses the implant 100, separating the top 132 of the implant 100 from the bottom 134 of the implant 100. In various examples, the vertical axis 130 can be perpendicular to at least one of the longitudinal axis 110 or lateral axis 120, the vertical axis can be substantially perpendicular to at least one of the longitudinal axis 110 or lateral axis 120, or the vertical axis can be askew to at least one of the longitudinal axis 110 or lateral axis 120.

As further illustrated in FIG. 1A, the anterior side 112 of the implant 100 can have an arc-shaped profile. The arc-shaped profile can also be referred to as a convex profile, a rounded profile, curved profile, or the like. The arc-shaped profile of the anterior side 112 can project at a first apex 116 located approximately equidistant between the right side 122 and the left side 124 of the implant 100. The arc-shaped profile of the anterior side 112 can terminate at each of a first end 126 and a second end 128 on the right side 122 and left side 124, respectively, of FIG. 1A. In an example, the first apex 116 can be equidistant between the first end 126 and the second end 128.

The posterior side 114 of the implant 100 can also have an arc-shaped profile similarly shaped to the arc-shaped profile of the anterior side 112. Again, the arc-shaped profile can also be referred to as a convex profile, a rounded profile, a curved profile, or the like. In an implanted position, the implant 100 can have a curved profile when it is against an anatomical surface. The curved profile of the posterior side 114 can project at a second apex 118 located approximately equidistant between the right side 122 and the left side 124. The arc-shaped profile of the posterior side 114 can terminate at each of a first end 126 and a second end 128, thereby meeting at the same points as the arc-shaped profile of the anterior side 112. In an example, the second apex 118 can be equidistant between the first end 126 and the second end 128.

FIG. 1B illustrates a top view of an alternative implant 150, having an anterior side 152 and a posterior side 154 in accordance with at least one example of the present disclosure. FIG. 1B illustrates the implant 150 which can be symmetrical about two axes, such as about the lateral axis 151 (e.g., separating the right side 155 from the left side 157) and a vertical axis 164 (e.g., separating the top side 165 from the bottom side 167). As illustrated in FIG. 1B, the implant 150 can have an asymmetrical profile about a longitudinal axis 160. The longitudinal axis 160 can represent the point, or plane, where the anterior side 152 transitions to the posterior side 154, or where the curve changes. Thus, the longitudinal axis 160 can separate the anterior side 152 from the posterior side 154. The longitudinal axis 160 can be between a first apex 156 on the anterior side 152 and a second apex 158 on the posterior side 154. The longitudinal axis 160 can also refer to or represent a lateral plane which traverses the implant 150, separating the anterior side 152 of the implant 150 from the posterior side 154 of the implant 150. The first apex 156 can project a greater distance from the longitudinal axis 160 than a distance from the longitudinal axis 160 to the second apex 158.

The longitudinal axis 160 can be off-center between the first apex 156 and the second apex 158. In an example, the posterior side 154 of the implant 150 can have a flatter arc-shaped, curved or rounded profile than the anterior side 152.

FIG. 1C illustrates a top view of an alternative implant 180, having an anterior side 182 and a posterior side 184 in accordance with at least one example of the present disclosure. FIG. 1C illustrates the implant 180 which can be symmetrical about two axes, such as about the lateral axis 192 (e.g., separating the right side 195 from the left side 197) and a vertical axis 194 (e.g. separating the top side 185 from the bottom side 187). As illustrated in FIG. 1C, the implant 180 can have an asymmetrical profile about a longitudinal axis 190. The longitudinal axis 190 can represent the point, or plane, where the anterior side 182 transitions to the posterior side 184, or where the curve changes. Thus, the longitudinal axis 190 can separate the anterior side 182 from the posterior side 184. The longitudinal axis 190 can be between a first apex 186 on the anterior side 182 and the posterior side 184. In the example illustrated in FIG. 1C, the posterior side 184 can be substantially flat or level. The posterior side 184 can be substantially or nearly parallel with a plane extending along the longitudinal axis 190. The longitudinal axis 190 can also refer to or represent a lateral plane which traverses the implant 180, separating the anterior side 182 of the implant 180 from the posterior side 184 of the implant 180. The first apex 186 can project a greater distance from the longitudinal axis 160 than a distance from the longitudinal axis 190 to the posterior side 184. The longitudinal axis 190 can be off-center between the first apex 156 and the posterior side 184. Such implants can be referred to as “flat backed” implants.

FIG. 1D illustrates an alternative implant 170 having a right side 171 and a left side 172. The implant 170 of FIG. 1D can be symmetrical about one axis, such as the lateral axis (e.g., separating the right side from the left side). The implant 170 of FIG. 1D can be asymmetrical about a longitudinal axis 175 and/or a vertical axis 176. The longitudinal axis 175 can separate the anterior side 177 from the posterior side 178. In an example, the posterior side 178 can be substantially flat such that the profile can be similar to a pectoralis muscle. In an example, the posterior side 178 can have a flattened curve shaped profile with a minimal apex 173. The anterior side 177 can have an apex 179 which can project at a location lower than the apex 173 on the posterior side 178. The vertical axis 176 can separate the top side 181 from the bottom side 191. The top side 181 can have a profile which narrows or decreases the further the implant 170 projects upward from the vertical axis 176. The bottom side 191 can widen or increase the further the implant 170 projects downward from the vertical axis 176. Such implants can be referred to as “teardrop” shaped implants.

Implants similar to any of the previously discussed shapes, profiles or forms can be used for body contouring implants such as breast implants, pectoral implants, gluteal implants, and facial implants without departing from the intended scope of the present disclosure.

In an example, the posterior side 114/154/174/184 of the implant 100/150/170/180, when inserted in a target site of the body, can flatten, conform or compresses against, for example, the pectoralis muscle or similarly located tissue in the breast area. When the posterior side 114/154/174/184 flattens, conforms or compresses, the anterior side 112/152/176/186 can expand or project into the breast tissue and away from the pectoralis muscle. This “volume” or “shape” shifting will be discussed in further detail below.

FIG. 1E is a chart illustrating exemplary projections for a series of implants similar to implant 180 which can have a “flat back”, as illustrated in the example implant shown in FIG. 1C, ranging in size from approximately 8.0 centimeters in diameter to approximately 15.0 centimeters in diameter. The implants 180 of FIG. 1E can be similar to the implants 180 discussed above which can be symmetrical about two axes, such as the lateral axis and vertical axis. The exemplary implant 180a illustrated in FIG. 1E can have a substantially flat posterior side 184a. The exemplary implant 180a illustrated in example A, can have a filler volume between approximately 135 cubic centimeters (hereinafter “cc”) and approximately 825 cc. The implant 180a of example A can have a base, or the posterior side 154a, which can be between approximately 8.0 centimeters and approximately 15.0 centimeters in diameter. In example A, the expansion or projection 183a, of the anterior side 182a can be between approximately 3.3 centimeters and approximately 6.1 centimeters as measured from the posterior side 184a to the anterior apex 156a.

The exemplary implant 180b illustrated in FIG. 1E can have a substantially flat posterior side 184b. In example B of FIG. 1E, the exemplary implant 180b can have an increased volume when compared to the volume of the exemplary implant 180a illustrated in example A. For example, the implant 180b in example B can have a filler volume between 160 cc and 1060 cc. The implant 180b, as illustrated in example B can have a base or posterior diameter similar to example A, which can be between approximately 8.0 centimeters and approximately 15.0 centimeters. The expansion or projection of the anterior side 182b can be between approximately 3.8 centimeters and approximately 7.8 centimeters as measured from the posterior side 184b to the anterior apex 186b.

FIG. 1F is a chart illustrating exemplary projections for a series of implants ranging in size from approximately 8.0 centimeters in diameter to approximately 15.0 centimeters in diameter. The implants 100 of FIG. 1E can be similar to implants 100 discussed above which can be symmetrical about three axes, such as the longitudinal axis, lateral axis and vertical axis. These exemplary implants can have a generally rounded posterior side 114 profile and a diameter between approximately 7.0 centimeters and approximately 10.0 centimeters.

In example C, as illustrated in the chart in FIG. 1F, the exemplary implant can have a filler volume between 100 cc and 190 cc. The implant can have an expansion or projection 133c of the anterior side 112c which can be between approximately 2.8 centimeters and approximately 3.4 centimeters as measured from the posterior apex 118c to the anterior apex 116c, when the implant is measured from a flat resting surface to the projection.

In example D, as illustrated in the chart in FIG. 1F, the exemplary implant can have a filler volume between approximately 120 cc and approximately 220 cc. The implant in example D can have an expansion or projection 133d of the anterior side 112d which can between approximately 3.3 centimeters to 4.0 centimeters as measured from the posterior apex 118d to the anterior apex 116d, when the implant is measured from a flat resting surface to the projection.

In example E, as illustrated in the chart in FIG. 1F, the exemplary implant can have a filler volume between approximately 135 cc and approximately 265 cc. The implant can have an expansion or projection 133e of the anterior side 112e which can be between approximately 3.7 centimeters and approximately 4.6 centimeters as measured from the posterior apex 118e to the anterior apex 116e, when the implant is measured from a flat resting surface to the projection.

In example F, as illustrated in the chart in FIG. 1F, the exemplary implant can have a filler volume between approximately 145 cc and approximately 290 cc. The implant in example F can have an expansion or projection 133f of the anterior side 112f which can be between approximately 3.9 centimeters and approximately 5.0 centimeters as measured from the posterior apex 118f to the anterior apex 116f, when the implant is measured from a flat resting surface to the projection.

One or more surfaces of the implants described herein (such as implant 100, 150, 170, 180, 200 or 1110) can have a surface texture (e.g., microtexture or nanotexture) to promote biocompatibility. For example, the implant can have a surface texture as disclosed in WO 2017/196973, WO 2015/121686, and/or WO 2017/093528, each incorporated by reference herein in its entirety. For example, the surface texture can include uniform surface features on the order of nanometers to micrometers.

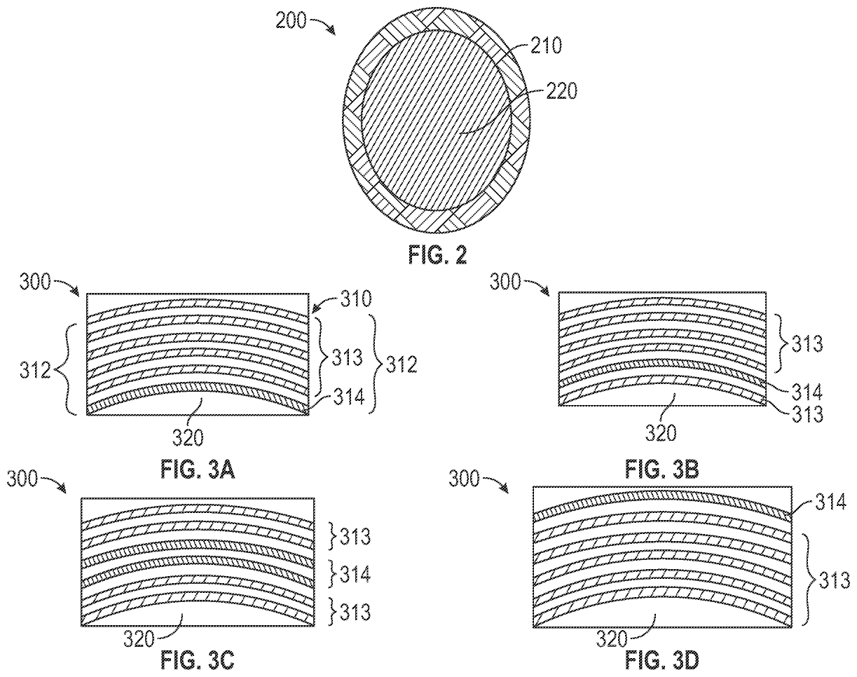

FIG. 2 illustrates a cross sectional view of an implant 200 in accordance with at least one example of the present disclosure. The implant 200 can comprise a shell 210 and a filler 220 (e.g., a fluid such as a gel or a liquid) inside a cavity formed by the shell 210. The shell 210 and the filler 220 can each comprise a biocompatible material, such as silicone. The filler 220 can be in the form of a fluid, e.g., a gel filler, having a viscosity or penetration value which can simulate movement of natural breast tissue. The penetration value is an example indication of the qualitative determination of the firmness of the filler 220. For example, the filler 220 can comprise a gel having a penetration value, defined as the vertical distance penetrated by the point of a standard needle and is usually measured in one tenths of a millimeter, ranging from about 10 tenths of a millimeter to about 60 tenths of a millimeter. The penetration value can be defined as a factor which measures the firmness of a colloid, such as a silicone gel. The penetration value can be measured according to ISO standard 14607 and/or ASTM F703. Any of the materials and/or features discussed in US Pat. No. 9,901,438, US Pat. Pub. No. 2015/0150675, and/or WO 2017/196973, each incorporated by reference herein in its entirety, can be used for the implants described herein. In an example, the filler 220 can be a viscoelastic silicone gel with superior adaptability capabilities in order to maintain high cohesivity and elasticity during any compression or force which the implant can be subject to. The higher viscosity in an example gel, the more natural the look of the breast can be after implanting the implant in the breast. In another example, a highly elastic gel which exhibits low rigidness can also give a more natural look to a breast after the implanting the implant. In an example implant surgical procedure where the implant is compressed or deformed while the implant is inserted into the breast, a highly elastic gel can allow the implant to return to an original form (e.g., the form of the implant when not being subjected to external stresses or forces), or close to an original form.

According to some examples herein, the shell 210 of the implant 200 can have a total thickness ranging from about 0.010 inches to about 0.040 inches, such as from about 0.013 inches to about 0.040 inches, from about 0.010 inches to about 0.020 inches, from about 0.012 inches to about 0.015 inches, from about 0.01 0inches to about 0.015 inches, from about 0.013 inches to about 0.025 inches, from about 0.015 inches to about 0.030 inches, or from about 0.020 inches to about 0.040 inches. The shell 210 can comprise at least one layer or a plurality of layers, e.g., having a total thickness of at least 0.01 inches, at least 0.012 inches, at least 0.01 5inches, at least 0.02 inches, at least 0.025 inches, at least 0.03 inches, at least 0.035 inches, or at least 0.04 inches. Elongation and ultimate break force of the shell can be measured according to Non-active surgical implants—Mammary implants—Particular requirements ISO 14607 or Standard Specification for Implantable Breast Prostheses ASTM F703-18.

FIG. 3A is a cross-sectional view of a portion of an implant 300 in accordance with at least one example of the present disclosure. As illustrated in FIG. 3A, the implant 300 can include a shell 310 and a filler 320. The exemplary implant 300 can be formed with at least one of the shell 210 or filler 220 characteristics described above. As further illustrated in FIG. 3A, the implant shell 310 can comprise a plurality of layers 312. For example, the shell 310 can comprise from 1 to 15 layers, from 2 to 8 layers, from 3 to 7 layers, from 4 to 15 layers, from 6 to 8 layers, from 10 to 13 layers, or from 12 layers to 15 layers. In some examples, one or more of the layers 312, or all of the layers 312, can comprise silicone.

According to some examples herein, the shell 310 can comprise at least two different types of layers, for example one or more standard shell layers 313 and one or more low diffusion barrier layers 314 having different chemical compositions. Each of the one or more standard shell layers 313 and each low diffusion barrier layer 314 can have the same thickness, or one or more layers can have a thickness different from one or more other layers of the shell 310. The shell 310 can include at least two low diffusion barrier layers 314. One or more low diffusion barrier layers 314 can be useful, for example, in implants intended to remain in the patient's body for a relatively long period of time. One or more low diffusion barrier layer 314 can, for example, inhibit leakage of materials within the implant 300 (e.g. in the filler 328) into the patient's body. However, implants which do not include a low diffusion barrier layer are also within the scope of the present disclosure. Exemplary implants which do not comprise a barrier layer can include, but are not limited to, sizers, tissue expanders, and tissue dissectors.

When present, the one or more low diffusion barrier layers 314 can be pigmented and have any desired color, such as blue, green, yellow, pink, orange, purple, or the like. In some examples, the one or more low diffusion barrier layers 314 can have the same color as other layers (including the one or more standard shell layers 313) present in the shell 310. Having a low diffusion barrier layer 314 which is pigmented differently than a standard shell layer 313 can assist a medical professional in detecting if there has been a rupture or defect in the implant 300 and/or confirming the implant 300 has been manufactured properly.

The one or more low diffusion barrier layers 314 can have a chemical composition different from the chemical composition of other layers 312 (such as the one or more standard layers 313). For example, the one or more low diffusion barrier layers 314 can comprise a silicone elastomer having a polysiloxane backbone and substituted or pendant functional groups which can inhibit permeation of silicone through the layer. The silicone elastomer can comprise polydimethylsiloxane. Exemplary functional groups include, but are not limited to, phenyl groups and fluorine groups. For example, the one or more barrier layers 314 can comprise silicone substituted with one or more diphenyl groups, methylphenyl groups, trifluorpropyl groups, and combinations thereof.

The one or more standard shell layers 313 can comprise silicone. In an example, the silicone can comprise polydimethysiloxane. When one or more standard shell layers 313 are present, the chemical composition of one or more of the layers can be different than the chemical composition of the other layer(s). In an example, the one or more standard shell layers 313 can comprise a high strength silicone dispersion of siloxanes and silicones, dimethyl vinyl group-terminated and silanamine, 1,1,1-trimethyl-N-, hydrolysis products with silica.

As depicted in FIG. 3A, the one or more low diffusion barrier layers 314 can form the innermost layer(s) of the shell 310, can be next to the filler 320, and the one or more standard shell layers 313 can form the outermost layer(s) of the shell 310. In particular, FIG. 3A depicts a single low diffusion barrier layer 314 which is in contact, such as direct contact, with one of the one or more standard shell layers 313 and the filler 320.

The position of the one or more low diffusion barrier layers 314 can vary. For example, FIG. 3B illustrates a single low diffusion barrier layer 314 disposed or sandwiched between adjacent standard layers 313. In other examples, as illustrated in FIG. 3C the shell 310 can include a plurality of low diffusion barrier layers 314 disposed or sandwiched between adjacent standard layers 313. In yet other examples, as illustrated in FIG. 3D, the one or more low diffusion layers 314 can form the outermost layer(s) of the shell 310. Numerous other orientations and compositions are discussed, for example, in U.S. Pat. No. 10,111,744, which is hereby incorporated by reference in its entirety.

### Marking Tool

FIG. 4 is a top view of a marking tool 400 in accordance with at least one example of the present disclosure. The marking tool 400 can be beneficial for a medical professional to use to better assist in correct location and placement of the implant. As will be discussed in further detail below, the marking tool 400 can provide a plurality of navigation or reference lines which a medical professional can use to mark or draw to indicate placement of medical tools or to indicate where procedures can occur. The marking tool 400 can also provide the medical professional a way to draw substantially similar, or identical, lines on the other breast.

The marking tool 400 can be sized and shaped according to the desired implant to be inserted. In the example illustrated in FIG. 4, the marking tool 400 can be made from a flexible material such as papers or plastics which can allow the marking tool 400 to contour to the shape of the breast. The marking tool 400 can be circular in shape and/or similar to the shape of the desired cavity to be formed in the patient's body. The marking tool 400 can have a center opening 402 in which the patient's nipple can be placed as a center point.

In the example illustrated in FIG. 4, the marking tool 400 can have at least one of the plurality of navigation lines or reference lines. Such reference lines can be at least one of a plurality of dimensional indicators 401. The at least one of the plurality of dimensional indicators 401 can correspond to different sizes of implants to be implanted. For example, at least one of the plurality of dimensional indicators 401 can be an outer perimeter 406 of the marking tool 400 which can correspond to a large implant. At least one of the plurality of dimensional indicators 401 can be an inner perimeter 422 which can correspond to a small implant. At least one of the plurality of dimensional indicators 401 can be disposed on the marking tool between the outer perimeter 406 and inner perimeter 422 as an intermediary marking area, representing a mid-sized implant.

In an example of the marking tool 400, an alignment portion 404 which extends from a first side 410 of the inner perimeter 422 of the marking tool 400 to a second side 412 of the inner perimeter 422 of the marking tool 400. The alignment portion 404 can have an extension 414 extending beyond the outer perimeter 406 of the marking tool 400. The extension 414 can provide an incision location identifier 405 or line pointed in the direction of the location of the incision. In an example, the extension 414 can orient the medical professional such that the incision can be cut in the axillary skin crease behind the contour of the pectoralis major muscle. The outer profile 413 of the extension 414 can be outlined, and a furthest point 415 can be marked as the location of the incision. The alignment portion 404 can provide a guiding line 416, as an example of a navigation line, for the surgeon to trace or mark which can extend between the center opening 402 and the point of the incision. In an example, the guiding line 416 can be an opening formed within the alignment portion 404. The guiding line 416 can extend from the center opening 402 toward the outer perimeter 406 of the marking tool 400. The point where the dimensional indicator 401 (such as outer perimeter 406, inner perimeter 422 or the intermediary marking area 424) and the guiding line 416 meet can indicate or identify a junction to mark as the final dissection mark 1416 (as discussed further below).

The outer perimeter 406 of the marking tool 400 can be a continuous form, such as a continuous circular shape, or other shape which corresponds to the implant to be inserted. In an example, as illustrated in FIG. 4, the outer perimeter 406 is a somewhat continuous circular profile. However, the outer perimeter 406 can be any shape as dictated by the use. The junction 417 of the extension 414 and the outer perimeter 406 can be indicated with a marking in, for example, the region known as the tail of Spence. The tail of Spence is an anatomical description for an extension of tissue of the upper lateral quadrant of the breast to the axilla area. In the illustration shown in FIG. 4, the outer perimeter 406 circular profile can represent the widest dimensional indicator (such as a diameter) on the marking tool 400 for marking the patient's body. In an example, the diameter can be approximately 10.0 centimeters, although various other diameters are possible as will be appreciated by those skilled in the art.

The profile of the marking tool 400 can provide another dimensional indicator which can also correspond to the implant to be inserted. For example, in a circular profile of the marking tool 400, the profile can be opened within an inner area 418 of the marking tool 400, such that the skin of the breast is exposed. The inner perimeter 422 of the inner area 418 can be a dimensional indicator for a specified dimension on the area of the breast. The inner perimeter 422 can be between approximately 6.0 and approximately 10.0 centimeters in diameter, such as approximately 8.0 centimeters. However, the inner area 418 can be a smaller cutout so long as there is enough space for a writing or drawing tool, such as a pen or marker, to be inserted and allow the medical professional to draw markings on the breast or other implant area.

Another area a medical professional can use as the dimensional indicator can be the intermediary marking area 424 which can be provided on the marking tool 400. The intermediary marking area 424 can correspond to the mid-sized implant. For example, the intermediary marking area 424 can be a specified distance removed from the center opening 402 and the inner perimeter 422. For example, the intermediary marking area 424 can be between approximately 7.0 and approximately 11.0 centimeters in diameter, for example approximately 9.0 centimeters.

The intermediary marking area 424 can include openings or slits in the marking tool 400 at the specified distance. The openings or slits of the intermediary marking area 424 can have a profile which allows a medical professional to accurately mark the distance at or around the target site. In the example illustrated in FIG. 4, the intermediary marking area 424 can be a series of openings or slits dispersed in an arcuate manner around the marking tool 400. In another example, the intermediary marking area 424 can be a somewhat continuous circle opening or slit in the marking tool 400.

While only one distance of an intermediary marking area 424 is illustrated in FIG. 4, there can also be more than one intermediary marking areas 424 such that the marking tool 400 can be used for different sized implants. For example, there can be a first intermediary marking area 424 approximately 8.5 centimeters, 9.0 centimeters, 9.5 centimeters in diameter, however these dimensions can vary.

### Cannula

FIG. 5 is a perspective view of a cannula 500 in accordance with at least one example of the present disclosure. The cannula can include an elongated shaft 501 and one or more openings or holes 510 in a distal end 502 of the shaft 501. The number of holes 510 can be dictated according to the purpose and the flow rate of the fluid intended to be dispensed from the cannula 500. For example, there can be approximately 1 hole to approximately 16 holes.

In an example, a cannula 500 with a shaft 501 having a diameter of approximately 1.8 millimeter to approximately 3.5 millimeters can be used. However, these dimensions can vary. A handle 512 can be located on a proximal end 504 of the shaft 501. The cannula 500 can be attached to an external fluid supply through the handle 512 or at any location along the cannula 500 as dictated by the use. The fluid can be, for example a saline-based anesthetic solution.

The fluid, in an example, can flow out of the shaft 501 through the holes 510 when the cannula 500 is inserted in designated locations around or proximate to the target site. A user can control the rate or amount of fluid being inserted with the handle 512 or another external control. In an example, the fluid flowing from the cannula 500 into the patient's body, when the patient's body is punctured by the cannula 500, can result in hydro-dissection.

### Tissue Dissector

FIG. 6 is a side view of a tissue dissector 600 (hereinafter, tissue dissector can include tissue separator) in accordance with at least one example of the present disclosure. The tissue dissector 600 is an example of an implement which can be inserted into the patient's body to assist in forming a cavity at the target site. The tissue dissector 600 can be configured for insertion through a relatively small incision, such as an incision of between approximately 2.0 centimeters and approximately 4.0 centimeters in length. Relatively small incisions can be of particular use during a minimally invasive procedure. Minimally invasive procedures can reduce damage to patient tissue at and around an implantation site or incision, thereby decreasing recovery time and reducing scarring at the incision site.

In some examples, the tissue dissector 600 can assist in the formation of a passage, tunnel, opening or canal extending from the incision to a target site for placing the implant. The tissue dissector 600 can have a distal end 602 designed to cut or separate layers of tissue, such as connective tissue, so a passage can be formed. The distal end 602 can terminate in a tapered profile 610 having a tip 612. In an example, the tip 612 can be dull. In another example, tip 612 can be sharp. The distal end 602 can be formed in any profile which allows a user to separate or cut through tissue within the patient's body.

The tissue dissector 600 can generally have an elongated profile with a center portion 620 which can be sized and shaped similar to the size and shape of the desired passage extending from the incision to the target site. In an example, the center portion 620 can be shaped in a substantially cylindrical profile. In another example, the center portion 620 can shaped in a tubular profile. The center portion 620 can have a maximum diameter between approximately 1.0 centimeter and approximately 4.0 centimeters, such as approximately 2.0 centimeters. The length of the tissue dissection 600 from the tip to a proximal portion of the center portion 620 can be between approximately 10 centimeters to approximately 30 centimeters.

The center portion 620, in an example, can have indexing markings 622. The indexing markings 622 can be formed on the outer surface of the center portion 620, or if the tissue dissector 600 is made from a transparent material, on the interior of the center portion 620. The indexing markings 622 can be painted, stamped, embossed, indented or the like on the surface of the center portion 620 or within the center portion 620. The indexing markings 622 can be placed at predetermined measurement points along the length of the center portion 620. For example, the indexing markings 622 can indicate distances in millimeters, centimeters, inches or the like. The indexing markings 622 can be a measurement from the tip 612 of the tapered profile 610. The medical professional can use the indexing markings 622 to determine the depth which the tissue dissector 600 has been inserted through the incision and thereby can indicate or can be a visual reference to the length of the passage the tissue dissector 600 has formed from the incision to the target site.

At the proximal end 604 of the tissue dissector 600 there can be a handle or a grip 630. The grip 630, in an example, can be formed in a manner which allows a medical professional to sufficiently hold and operate the tissue dissector 600. The grip 630 can be formed with non-slip features such as ribs, grooves, protrusions or the like to assist the medical professional manipulating the tissue dissector 600 through the tissue without causing the tissue dissector 600 to slip during use. In another example, the grip 630 can be covered with a material which can have nonslip functionalities. The grip 630, in an example, can be formed in a manner which allows a medical professional to efficiently use the tissue dissector 600 to separate or cut the tissue in the patient's body so a passage can be formed. In an example, the grip 630 can have a bulbous, rounded or other shape which can be ergonomically designed to fit within a user's hand.

### Tissue Expander

FIG. 7A is a perspective view of a tissue expander 700 in accordance with at least one example of the present disclosure. Tissue expander can refer to any device or implant that when expanded in size can separate layers of tissue or form gaps or spaces between layers of tissue. As illustrated in FIG. 7A, the tissue expander 700 can include a balloon 750, a flexible lumen 720, an adapter 722 or other connection element, and a pump 710 configured to be connected to the adapter 722. A tissue expander 700 can be a device which expands, inflates or otherwise increases in size to separate or form a cavity within at least two layers of tissue, ligaments, muscle, or the like.

The balloon 750 can be an inflatable and deflatable balloon and can be positioned at a distal end 719 of the flexible lumen 720 and the pump 710 can be positioned at a proximal end 724 of the lumen 720. The pump 710 can be a unidirectional pump. In an example, when a first side 711 of the pump 710 is coupled to the proximal end 724 of the lumen 720, utilizing the pump 710 can inflate the balloon 750 connected to the pump 710 via the flexible lumen 720. In particular, when the first side 711 of the pump 710 is coupled to the flexible lumen 720, actuating the pump 710 can inflate the balloon 750. Attaching a second end 713 of the pump 710 to the flexible lumen 720 can deflate the balloon 750 when the pump 710 is actuated.

In an example, the pump 710 can be a manual bulbous-shaped pump which the user can actuate by applying a compressive force to the pump 710. Pressurized air can be forced through the flexible lumen 720 to and/or from the balloon 750, thereby inflating or deflating the balloon 750. In another example, the pump 710 can be an electronically controlled mechanism which the medical professional can activate or control and which can cause fluid or air to be transmitted to and/or from the balloon 750 thereby inflating or deflating the balloon 750.

A clip 730 can be attached to the flexible lumen 720 to control the flow of fluid or air through the flexible lumen 720. In an example, the clip 730 can be a Luer lock fitting, a clamp or other similar mechanism to control fluid flow. In an example, the clip 730 can be closed when the balloon 750 has been expanded to a desired size. Closing the clip 730 can prevent the air, pressurized air, or other pressurized fluid from being expelled from the expanded or inflated balloon 750. The air, pressurized air, or other pressurized fluid can be released from the balloon 750 by removing or opening the clip 730, thereby deflating the balloon 750.

The balloon 750 of the tissue expander 700 can have a circular or other generally rounded shape when deflated, and a spherical, ellipsoid, ovoid, or other generally rounded shape when expanded. It will be understood, however, any shaped balloon 750 can be used for the expandable implement or device. In some examples, the balloon 750 can be shaped to form a desired cavity within the dissected tissues depending on a desired procedure. The balloon 750 can be inflated to expand in both the posterior side and the anterior side. The balloon 750 can be inflated such that the anterior side projects further from a center point of the inflated balloon than the posterior side projects from the center point. The balloon 750 can have a rounded posterior and/or a rounded anterior side. In an example, the balloon 750 can form a nest-like cavity within the target site, as discussed further below. A rounded shape balloon can be suitable, for example, for a target site in which a rounded implant is intended to be inserted.

The balloon 750 can have any suitable thickness and size. In some examples, the balloon 750 can have a wall thickness of between about 0.1 millimeters and about 0.5 millimeters, such as about 0.1 millimeters, about 0.15 millimeters, about 0.2 millimeters, about 0.25 millimeters, about 0.3 millimeters, or about 0.35 millimeters. In some examples, the balloon 750 can have an uninflated diameter of between approximately 10.0 centimeters and approximately 20.0 centimeters, such as between approximately 10.0 centimeters and approximately 15.0 centimeters, such as approximately 12.0 centimeters.

At least one of the balloon 750 and the flexible lumen 720 disclosed herein can be made of a flexible biocompatible polymer, such as silicone. In some examples, the balloon 750 and flexible lumen 720 can be made from a single material, and in alternate examples, can be made from different materials. The flexible lumen 720 can extend from an outer wall of the balloon 750 and can be in fluid communication with an interior of the balloon 750. The flexible lumen 720 can be of any suitable length, such as, e.g., between about 20.0 centimeters and about 50.0 centimeters. The length of the flexible lumen 720 can vary to ensure the flexible lumen 720 extends from the balloon 750 positioned in the patient's body cavity to the exterior of the patient.

As illustrated in FIG. 7A, the tissue expander 700 can include an introducer 740. The introducer 740 can be disposed between the pump 710 and the balloon 750. The introducer 740, in an example, can be a hollow tube or tube-like component through which the flexible lumen 720 can traverse. The length of the introducer 740 can be a length similar to the tissue dissector 600 or shorter than the length of the tissue dissector 600. In an example, the introducer 740 can have indexing markings 741. The indexing markings 741 can be at regular intervals on an exterior surface or an interior surface of the introducer 740. The indexing markings 741 should be visible to the medical professional. The indexing markings 741 can be measured from a tip 746 of a supporting extension 745. The indexing markings 741 can be used by a medical professional to determine how far the introducer 740 has been inserted into the passage extending from the incision to the target site. The indexing markings 741 can indicate to a medical professional when the balloon 750 is at the target site. The indexing markings 741 can be placed in predetermined measurement points along the length of the introducer 740. For example, the indexing markings 741 can indicate distances in millimeters, centimeters, inches or the like.

A proximal end 744 of the introducer 740 can include a handle 742. The handle 742 can be a bulbous shape, rounded shape or any other ergonomically designed form. The medical professional, in an example, can grip or hold the introducer 740 by the handle 742 when inserting the introducer 740 and the balloon 750 into the incision and passage. In an example, the flexible lumen 720 can enter a first opening 741a of the handle 742.

A distal end 743 of the introducer can include a second opening 743a through which the flexible lumen 720 can pass and exit the introducer 740. The flexible lumen 720 can be coupled with the balloon 750 at the second opening 743a or the flexible lumen 720 can extend past the second opening 743a a predetermined distance and the balloon 750 can be coupled at the distal end 721 of the lumen away from the introducer 740.

In an example, the introducer 740 can have a supporting extension 745 protruding from the second opening 743a. The supporting extension 745 can extend from a bottom side 743b of the second opening 743a when the introducer is in an orientation to be inserted into an incision. In an example, the supporting extension 745 can have a tip 746, as illustrated in FIG. 7B, which can bisect tissue in the target site. The supporting extension 745 can be made of any material which can bisect or separate tissue without being damaged during the insertion of the supporting extension 745 and the coupled balloon 750 through the passage formed by the tissue dissector 600.

The balloon 750 can be coupled to the supporting extension 745 such that the balloon 750 can be supported when the balloon 750 is in inflated or expanded, or deflated or contracted. The supporting extension 745 can be coupled with a posterior side 751 of the balloon 750 with an adhesive material, straps such as silica straps, or with a mechanical coupling such as a snap fitting coupling. The supporting extension 745 can remain substantially stationary relative to the balloon 750 when expanding or contracting the balloon 750. Before the balloon 750 can be introduced into the incision, the balloon 750 can be contracted, such as, folded, rolled, wrapped or otherwise condensed in size around or on the supporting extension 745 such that it is a size to fit in the incision. For example, the width of the balloon 750 in the compressed form can be between approximately 1.0 centimeter and approximately 4.0 centimeters. In another example, the width of the balloon 750 in the compressed form can be between approximately 1.0 centimeter and approximately 3.0 centimeters. In another example, the width of the balloon 750 in the compressed form can be between approximately 1.0 centimeter and approximately 2.0 centimeters.

The length of the supporting extension 745 can be dictated by the size of the balloon 750 used. In an example, the supporting extension 745 can be longer in length than the diameter of the balloon 750. In another example, the supporting extension 745 can be substantially equivalent in length or less than the length relative to the diameter of the balloon 750.

In an example, the posterior side 751 of the balloon 750 can be coupled with the supporting extension 745 such that at posterior side 751 where the balloon 750 can couple with the supporting extension the balloon 750 can be held substantially flat while the outer perimeter of the posterior side 751 can have a curved, or slightly curved, profile. The balloon 750 can be supported or braced such that the balloon 750 can remain stationary during expansion of the balloon 750 to an inflated state. The balloon 750 can project from the supporting extension 745 and expand into the tissue at the target site.

### Injector

FIG. 8 is an exploded, perspective view of an injector 800 that can be used to insert an implant (such as implants 100, 150, 170, 180, 200, 1110) into a cavity formed at the target site in accordance with at least one example of the present disclosure. In some examples, the injector 800 can be configured for one-handed advancement of the implant into the target site. The injector 800, as illustrated in FIG. 8, can include a nozzle 810 and a handle 820. The nozzle 810 can define a housing 910 configured to hold an implant and can include a nozzle end 812 having a nozzle end opening or distal opening 912, a middle portion 911, and a nozzle engagement area 816 for engaging with a complementary handle engagement area 826 of the handle 820. The handle 820 can further include a fluid supply conduit or lumen 830 and an actuator 840.

The injector 800 can have any of a variety of suitable sizes, shapes, and characteristics suitable for holding and delivering an implant. In some examples, the injector 800 can be a single-use (e.g., disposable) device, or some or all of the injector 800 (e.g., the handle 820 and/or the nozzle 810) can be reusable, such as after sterilization.

The nozzle 810 can be a single piece, or can comprise multiple pieces which can be fitted, slotted, assembled, clipped, welded, or otherwise joined together at one or more joining points. The nozzle 810 also can have additional profiles and features (e.g., with respect to the housing 910 for holding an implant and/or with respect to the nozzle end opening 912), as described further herein. The nozzle 810 can be formed from or can otherwise comprise one or more biocompatible polymer or copolymer material(s). Exemplary materials can include polyurethane, polyethylene, silicone, polycarbonate, or a combination thereof. The nozzle 810 can be rigid, semi-rigid, flexible or a combination thereof. For example, the nozzle end 812 of the nozzle 810 can be rigid enough to dilate the incision, but not widen the incision, and direct the implant to the incision site, but soft enough to avoid tearing or damaging the site and/or to avoid deformation of the implant. Moreover, the nozzle end 812 can be more flexible than, e.g., the nozzle engagement area 816, which can be more rigid to facilitate engagement with the handle 820.

The nozzle end 812 can be sized such that it extends and fits through the passage formed by the tissue dissector 600. The nozzle end 812 can be, for example, between approximately 5.0 centimeters and approximately 15.0 centimeters in length. The nozzle end 812 can be an aperture at or near a distal portion 923 of the middle portion 911 of the nozzle 810, through which the implant housed in the housing 910 of the injector 800 can exit the nozzle 810 during an implantation surgical procedure. In an example, the distal portion 923 including the nozzle end 812 can be connected (either in a solitary molded nozzle or component assembled nozzle) to the middle portion 911, including the housing 910 with a tapered area 914. The nozzle end 812 can be, in an example, an elongated cylindrical chamber extending from the tapered area 914 of the nozzle 810 to the nozzle end opening 912. The nozzle end 812 and the tapered area 914 can be any suitable length such that the implant) can be accurately placed within the target site with minimal subsequent work by the medical professional.

The tapered area 914 can assist in reducing any stresses applied to the implant 100 as the implant 100 can be expelled from the middle portion 911. The tapered area 914 can decrease in diameter from the middle portion 911 to the nozzle end 812. The implant can be further compressed when the implant enters the nozzle end 812 through the tapered area 914.

The middle portion 911 of the nozzle 810 can define the housing 910, and can be configured to be loaded with, and house or hold, the implant in a radially compressed and/or elongated configuration for introduction into a target site. In some examples, as shown, the distal portion 923 of the nozzle 810 can be more tapered (have a smaller cross-sectional dimension) than the middle portion 911, such that the implant loaded into the middle portion 911 is not as compressed as it would be in nozzle end 812. In some examples, the middle portion 911 can have an approximately equal diameter along its length. In some examples, for example, the middle portion 911 can be generally cylindrical in shape. The nozzle engagement area 816 of the nozzle 810 can be opened to allow for loading of an implant into the housing 910 defined by the middle portion 911.

FIG. 9 is a cross-sectional view of the injector 800 in accordance with at least one example of the present disclosure. As described above, the nozzle 810 can define the housing 910 for holding an implant in a compressed, rolled, or an otherwise compacted configuration prior to implantation. The dimensions of the nozzle 810 can be selected based on the dimensions (e.g., size and shape) of the implant to be delivered using the injector 800, and/or vice-versa (e.g., characteristics of the implant can be selected based on the dimensions of the nozzle 810).

A cross-sectional size of the nozzle end 812 can have any suitable size, to allow the implant to pass through. In some examples, a cross-sectional dimension of the nozzle end 812 can range from approximately 0.5 centimeters to approximately 5.0 centimeters, from approximately 1.0 centimeter to approximately 3.0 centimeters, or from approximately 1.0 centimeter to approximately 2.0 centimeters.

Any one or more portions of the nozzle 810, such as an inner surface 918 of the nozzle 810, can include a lubricious coating to reduce the coefficient of friction between one or more portions (e.g., the inner surface) of the injector 800 and one or more portions of the implant housed within. For example, a lubricious coating can be a water-activated coating fixed on one or more inner surfaces 918 of the nozzle 810. Additionally or alternately, a lubricious coating can include a biocompatible lubricant and/or any other biocompatible coating. The coating can reduce a coefficient of friction between the implant shell (such as shell 210 or 310) and the interior surface 918 of the nozzle 810, promoting a smooth transition between the insertion configuration and the deployed configuration of the implant, e.g., upon exit of the implant from the injector 800.

The nozzle 810 can be designed to reduce the risk of tearing or other damage to the implant or patient tissue. The nozzle 810 can be designed to aid in achieving a desired expulsion pressure against the implant when the injector 800 deposits the implant. The nozzle 810 can aid in achieving a desired ejection speed of the implant through the nozzle end opening 912 of the nozzle 810. In some examples, characteristics of, e.g., the nozzle end opening 912 can be designed or selected to achieve a desired implant ejection speed or implant ejection pressure, or can be designed or selected to improve placement precision of the injector 800, biocompatibility of the injector 800 with patient tissue, compatibility with a particular incision size, and/or other goals.

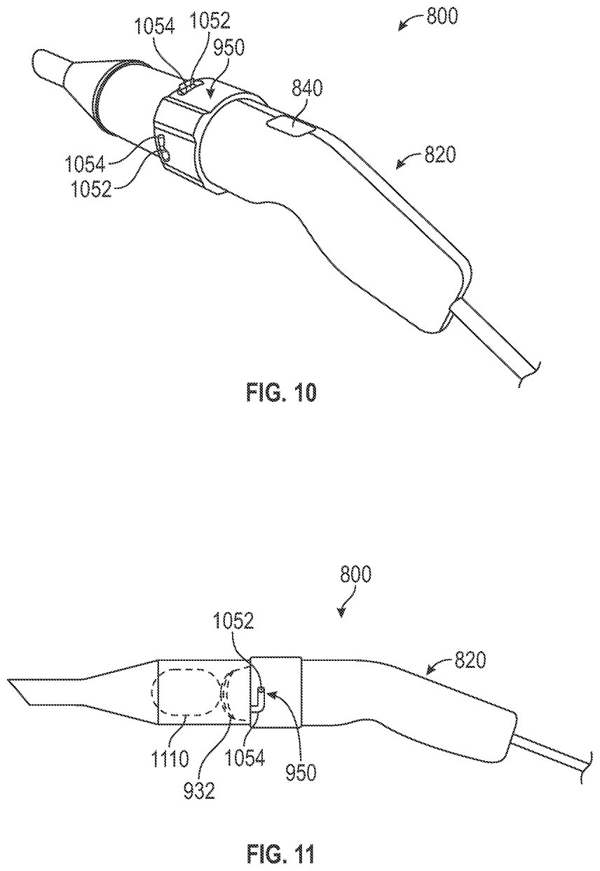

As illustrated in FIGS. 10 and 11, the handle 820 can be configured to be attached, detached and/or reattached to the nozzle 810 via a suitable mechanism. FIGS. 10 and 11 illustrate perspective and side views, respectively, of the injector 800 depicting an attachment mechanism 950 in accordance with at least one example of the present disclosure. The attachment mechanism 950 can include components which can be disposed at, on, and/or around the handle engagement area 826 of the handle 820 and/or the nozzle engagement area 816 of the nozzle 810. In some examples, the handle engagement area 826 can include a plurality of threads complementary to a plurality of threads of the nozzle engagement area 816. In further examples, the handle engagement area 826 and the nozzle engagement area 816 can include other mating features (e.g., clips, protrusions and recesses, clamps, adhesive, etc.) to facilitate attachment of the handle 820 to the nozzle 810 either permanently or reversibly.

As illustrated in FIG. 10, sliding protrusions 1052 can be provided on the nozzle 810 that are configured to fit into channels 1054 on the handle 820. Each protrusion 1052 can slide into a circumferential portion of the channels 1054 to secure the nozzle 810 to the handle 820. The channels 1054 can be of any shape, for example, the channels 1054 can have an L-shape, such that rotating the nozzle 810 relative to the handle 820 can lock the protrusions 1052 within the channels 1054. In an example, when the sliding protrusions 1052 reach a termination sliding point of the channel 1054, the engagement (for example the protrusion 1052 engaging the channel 1054) can lock the nozzle 810 with the handle 820. In another example, when the sliding protrusion 1052 reaches the termination sliding point of the channel 1054, the engagement can create an audible noise which can indicate to the medical professional the nozzle is secured with the handle 820.

The handle 820 and the nozzle 810 can be coupled in a manner such that a seal is formed within the nozzle engagement area 816. For example, the nozzle engagement area 816 and the handle engagement area 826 can join together with a friction fit and the attachment mechanism 950 such that a fluid impermeable seal can be formed between the handle 820 and the nozzle 810. The fluid impermeable seal can be coupled, placed, formed or otherwise attached proximate to the distal end of the handle engagement area 826. The fluid impermeable seal can also be coupled, placed, formed or otherwise attached proximate to the proximal end of a nozzle engagement area 816.

The handle 820 can define or encompass a fluid supply lumen 830, which can be configured for the passage of a fluid, e.g., from a fluid source (not shown). The fluid supply lumen 830 can be connected on one end with the fluid source through the handle 820 and to a fluid supply mouth 924. When the actuator 840, such as a button, is pressed, it activates fluid supply mouth 924, allowing the air continue flowing inside to the expandable chamber 930. The fluid supply lumen 830 can be coupled or couplable to a fluid supply via any suitable connection, such as, but not limited to, a Luer connection, threaded connection, clip connection, lock connection, etc. The fluid supplied can include a pressurized fluid source, such as a pressurized gas or liquid. In some examples, the pressurized fluid source can include, e.g., a portable compressed fluid canister, a pressurized fluid line (e.g., a gas line or water line), or the like. In some examples, the fluid source can be a disposable or refillable canister of compressed gas. In some examples, the fluid supply lumen 922 and the fluid supply mouth 924 can be configured to transfer pressurized fluid from the fluid supply lumen 922 into an expandable membrane chamber 930 or cavity defined by an expandable membrane 932 (e.g. balloon, diaphragm), coupled with the distal end of the handle 820. When the handle 820 is coupled with the nozzle 810, the expandable membrane 932 can be disposed at least partially within, adjacent, or proximate to the nozzle engagement area 816 of the nozzle 810. Pressure from pressurized fluid into the expandable membrane chamber 930 can expand the expandable membrane 932, balloon wall, or cavity wall to impart pressure on the implant and drive the implant distally. As described elsewhere herein (e.g., with respect to injector 800), the handle 820 can further include or be connected to a pressure regulator 860, which can allow for venting of pressurized fluid from the expandable membrane chamber 930.

A vent switch 842 can be disposed on the handle 820 which can control a vent fluidly coupling an interior of the nozzle 810 with an exterior of injector 800. According to some aspects, when vent switch 842 is closed, vent switch 842 prevents fluid from escaping from the expandable membrane chamber 930. Further, for example, when actuated or opened, vent switch 842 may allow for fluid within expandable membrane chamber 930 to vent outside of the injector 800, thereby deflating or reducing fluid pressure within expandable membrane chamber 930 to an extent that expandable membrane chamber 930 is pressurized relative to an exterior of the injector 800. Vent switch 842 may operate mechanically or electronically. In some embodiments, for example, vent switch 842 may include a powered switch that may, e.g., activate suction, a fan, or a blower to actively remove fluid from within expandable membrane chamber 930. Vent switch 842 may thereby be used to stop or reduce expulsion pressure within nozzle 810, e.g., to stop or slow expulsion of an implant from nozzle 810, and/or to reset the injector 800 after an implant has been expelled from nozzle 810.

The handle 820 can further include an actuator 840 for selectively supplying and terminating the flow of compressed gas or other pressurized fluid from the fluid supply through fluid supply mouth 924. The actuator 840 can include, e.g., a button, knob, valve, switch, clip, or combinations thereof, which can open/create and/or close a connection between a more proximal portion of fluid supply lumen 922 and the fluid supply mouth 924. In some examples, the actuator 840 can be spring-loaded or otherwise can employ consistent pressure to maintain an open flow of pressurized fluid towards an implant housed in the housing 910 of the nozzle 810. In an example, the actuator 840 can be located on an upper or top surface of the handle 820 such that a medical professional can easily access the actuator 840 while preventing accidental triggering or activating.

The implant 1110, as illustrated in FIG. 11, can be any implant previously described in the present disclosure or any similar implant which can be suitable for the dictated purpose. An implant (such as an implant 1110) can be loaded through the nozzle engagement area 816 of the middle portion 911 before the handle 820 is coupled to middle portion 911. FIG. 11 illustrates the expandable membrane 932 in a compressed, or deflated, configuration with the implant 1110 within the housing 910. The implant 1110 can be inserted into the housing 910 of nozzle 810 of the injector 800 by compressing the implant 1110. For example, the implant 1110 can be pre-loaded or inserted into the middle portion 911 to facilitate the sterile loading of the implant into the nozzle, and/or to manipulate (e.g., compress, elongate, etc.) the implant 1110 toward the insertion configuration.

A vacuum or suction can be used to load the implant 1110 into the housing 910. For example, a vacuum cup 960 (see FIGS. 8 and 9) can be affixed over the nozzle opening 912, e.g., to form a fluid-tight seal. A vacuum can be applied through a vacuum opening 962 of vacuum cup 960. The vacuum can be supplied via an external vacuum or suction-generated source. The vacuum source can be an in- room generated source, a portable source or any similar source of negative airflow designed for the desired purpose. The vacuum source can be any vacuum source known for such procedures as liposuction, endoscopy or the like. For example, a vacuum pressure of between approximately 508.0 to approximately 762.0 mmHg (approximately 20.0 to approximately 30.0 inHg) can be supplied through a lumen to the vacuum opening 962 and the vacuum cup 960. The implant 1110 can be placed at or near an open proximal end of nozzle 810. The reduction in pressure which can be caused by the applied vacuum can draw the implant 1110 into the proximal opening of the nozzle 810, and into the housing 910 for example, into the middle portion 911 of the nozzle 810.

An electrical supply conduit 970 (see FIG. 9) can supply electrical power to one or more aspects of the injector 800. In some examples, the electrical supply conduit 970 can supply electrical power to a vacuum source disposed in the handle 820, which can be used to create suction in, and load implant 1110 into the middle portion 911 of the nozzle 810. However, in other examples, the electrical system can be an internal component.

Once the implant 1110 has been loaded into the injector 800, pressurized fluid can be delivered through the fluid supply lumen 922 and the fluid supply mouth 924 to a region located proximally from the implant 1110. Such pressurized fluid, when delivered, can impart pressure on the implant 1110 to drive the implant 1110 distally towards and through nozzle end opening 912 of the nozzle 810. The pressure generated in the expandable membrane chamber 930 can expand and/or move the expandable membrane 932, balloon wall, or cavity wall in a distal direction to impart pressure on the implant 1110 and drive it distally, through the distal opening 912.

In some examples, the nozzle end opening 912 can have an angular profile such that an upper portion 915 of the nozzle end opening 912 extends further from the nozzle end 812 than a lower portion 917 of the nozzle end opening 912. The angular profile of the nozzle end opening 912 can allow the implant to be drawn, or pulled, from the nozzle 810 during the medical procedure by providing a larger effective diameter at the nozzle end opening 912.

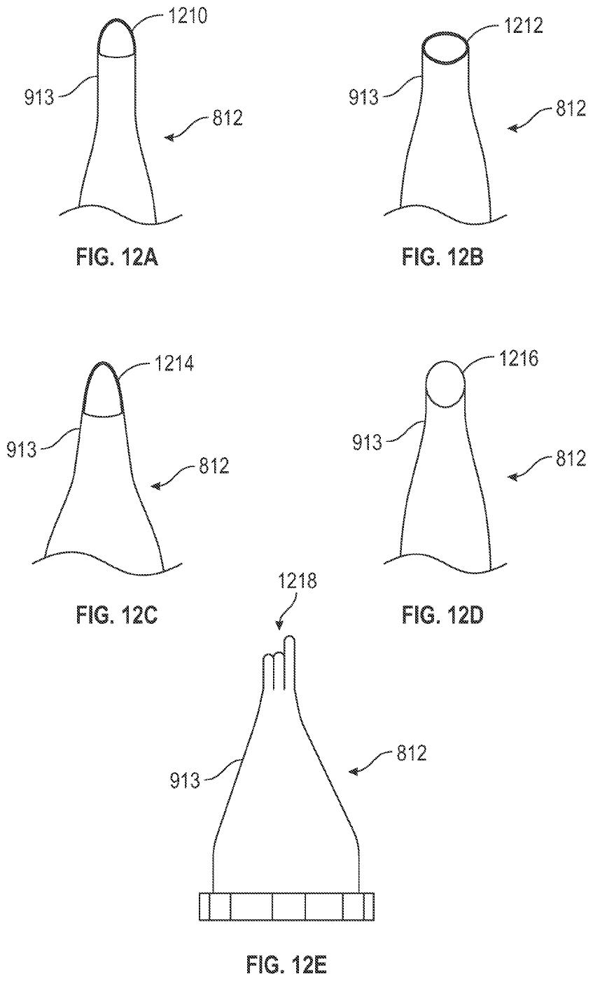

The nozzle end opening 912 can be a distal-most portion of the nozzle 810. FIGS. 12A-12E are top views of nozzle end openings 1210, 1212, 1214, or 1216 in accordance with various examples of the present disclosure. The nozzle end opening 912 can have any configuration suitable for depositing the implant (such as implants 100, 150, 200 or 1110) in the target site, e.g., as described herein and/or in WO 2017/181144, incorporated by reference herein in its entirety. The nozzle end opening 912 is at a distal end of a nozzle end 913 can have a perimeter dictated by the purpose or the profile of the implant. For example, the nozzle end opening 912 can have a half-oval or angular shape to accommodate a non-round implant. An angling of the nozzle end opening 912, and/or diameter of the nozzle end opening 912, can also be customized. The nozzle end opening 912 can have different exemplary shapes such as those associated with the nozzle end openings 1210, 1212, 1214, and 1216. For example, the perimeter of the nozzle end opening 912 can be generally rounded, circular, oval, oblong, trapezoidal, or asymmetrical in shape. The nozzle end opening 912 can have a larger diameter or cross-sectional dimension at its proximal end portion than the diameter or cross-sectional dimension at its distal end portion.

As illustrated in FIG. 12E, the nozzle end opening 1218 can be bordered, flanked, and/or defined by one or more slits, flaps, petals or extensions disposed about a perimeter of the nozzle end opening 1218. Such features can be disposed in a circumferential arrangement about the nozzle end opening 1218 or can be disposed symmetrically or asymmetrically about the nozzle end opening 1218. In some examples, such features can assist in positioning the nozzle end 812 through an incision and/or guiding placement of the implant through the nozzle end 812 and the nozzle end opening 912 into an implantation site.

In some examples, the nozzle can have tapered profile of different sized tapers, as illustrated in FIGS. 12A to 12E. For example, a relatively smaller percentage of the nozzle end opening 912 can include a tapered profile (e.g., between about 50%, to about 25% of the nozzle end opening 912 can have a tapered profile, or less if the design dictates). In some examples, features of the nozzle end opening 912 can be flexible (e.g., flexible enough to bend upon pressure being exerted on them by the passage of an implant, or, in some examples, more flexible than a proximal region of the nozzle 810). At least a portion of the nozzle 810, such as a perimeter of the nozzle end 812, can be configured to flex, e.g., as the implant passes through the nozzle end opening 912 of the nozzle 810. In another example, the cross-sectional size of the nozzle end 812 can increase as the implant passes therethrough (e.g., increasing from approximately 0.5 centimeters to approximately 2.0 centimeters, to approximately 2.5 centimeters, to approximately 3.0 centimeters, or to approximately 3.5 centimeters). In another example, the cross-sectional size of the nozzle end opening 912 can remain static as the implant passes therethrough.

### Surgical Method

A surgical method such as a minimally invasive surgical procedure can be used to insert an implant into a target site in a patient's body. While an implant can be placed within a target site in any known location, such as at the calves or around the gluteal muscles, a surgical method or procedure for placing an implant in a patient's breast area will be described further. Furthermore, although the surgical method or procedure will be described with reference to an implant 1110, those skilled in the art will appreciate the surgical method described herein can also be used for the implantation of any of the implants described above (such as implant 100, 150 170 or 200), or any known implant. Similar surgical tools used in the below described methods can be applied to gluteal implants, calf implants or other implants.

In an example, the below-described minimally invasive surgical procedure can be a monitored, conscious-sedation surgical procedure. In an example, the below-described, minimally invasive surgical procedure can utilize a local anesthetic.

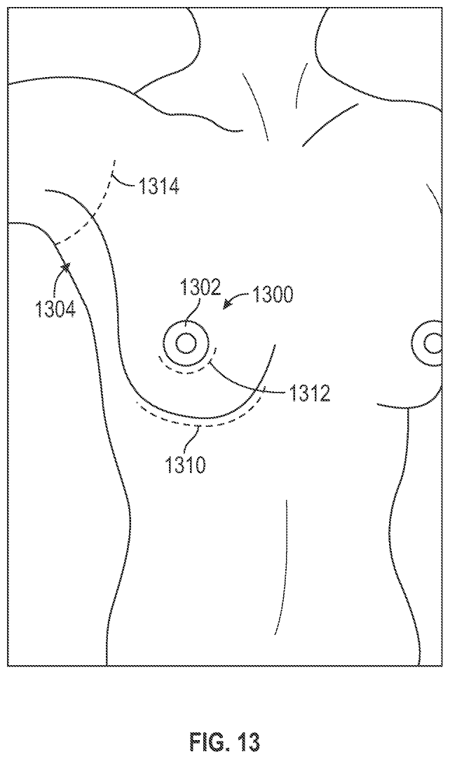

FIG. 13 illustrates a front view of a patient's torso as discussed in the present disclosure. A small incision can be made at a desired location such as an inframammary incision, a periaeolar incision or an axillary incision when the implant can be inserted in the breast 1300. As illustrated in FIG. 13, an inframammary incision 1310 can be made underneath a breast 1300. A periareolar incision 1312 can be made around an areola, or nipple, 1302. An axillary incision 1314 can be made in or proximate to an axilla area 1304.

While a minimally invasive surgical procedure where an incision can be made such as the inframammary incision 1310 or the periaeolar incision 1312 is contemplated, a minimally invasive surgical procedure utilizing an exemplary axillary incision 1314 will be described. However, the concepts and methods described for the axillary incision 1314 can also be applied to incisions made in the inframammary area or the periaeolar area.

### Use of Marking Tool

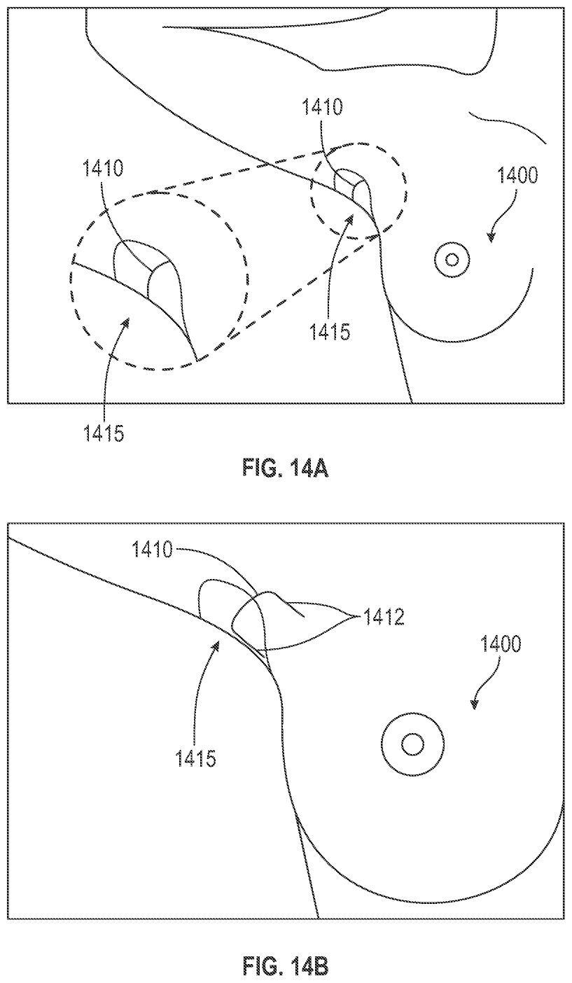

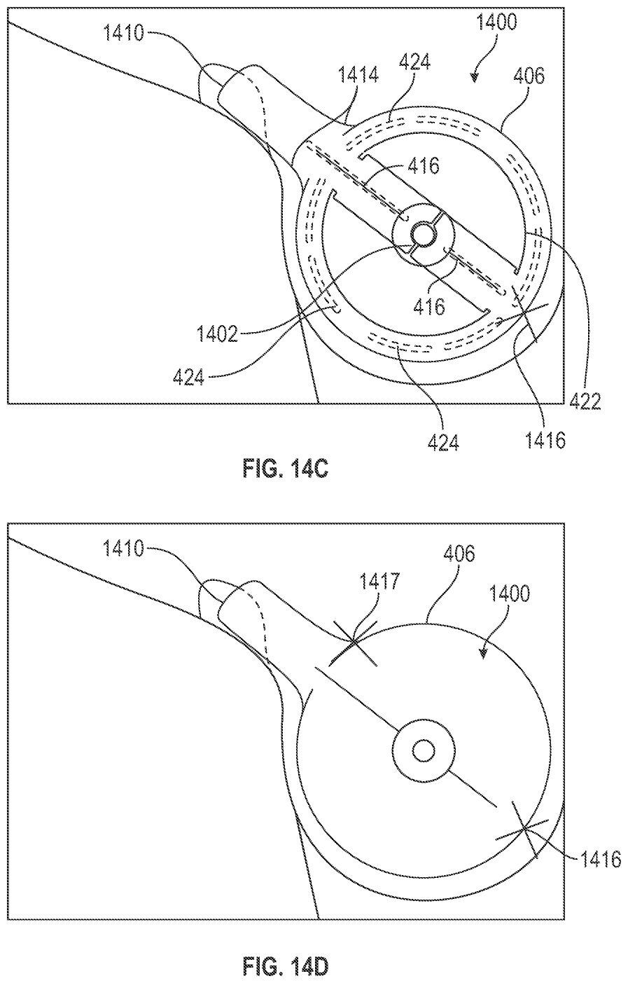

FIGS. 14A-14D illustrates several exemplary images of steps in a surgical procedure utilizing a marking tool in accordance with at least one example of the present disclosure. The marking tool 400, as described in relation to FIG. 4, or any other suitable device can be used to mark the surgical area using at least one of a plurality of navigation lines or reference lines on a breast 1400. In an example, the marking tool 400 can be placed on the breast 1400, with a center of the marking tool 400 substantially aligned with the areola 1402, or nipple, before or after an incision mark 1410 indicating the incision site is drawn, as illustrated in FIG. 14A.

The incision mark 1410 can be marked on the breast by tracing or drawing a line at an incision location identifier 405. The incision mark 1410 indicating the location for the incision can be drawn on a pre-existing axillary skin crease 1415 such as behind the contour of the pectoralis major muscle. The incision mark 1410 can extend along the axillary skin crease 1415 a length between approximately 1.0 centimeter and approximately 4.0 centimeters, for example approximately 2.5 centimeters.

The medical professional can use the marking tool 400 to mark parallel lines 1412 extending from lateral ends of the incision mark 1410, as illustrated in FIG. 14B. Alternatively, the medical professional can free-hand the parallel lines 1412. The parallel lines 1412 can be parallel to the muscle fiber orientation. The parallel lines 1412 can extend, for example, between approximately 3.0 centimeters and approximately 5.0 centimeters from lateral ends of the incision mark 1410 towards the breast 1400.

As illustrated in FIG. 14C, the medical professional can place the marking tool 400 on the breast 1400 with the patient's nipple or areola 1402 as the center point. The medical professional can use a pen, marker or other writing tool to mark, trace along or draw at least one of the plurality of reference lines corresponding to a dimensional indicator, such as a circumferential outline of the implant to be inserted. The circumferential shape can be the outer perimeter 406 of the marking tool 400 on the breast 1400. The outer perimeter 406 of the marking tool can relate to the target site of the implant. The medical professional can trace or draw markings at intermediary marking areas 424 or an inner perimeter 422 on the marking tool 400 when, for example, a smaller implant is to be inserted. The outer perimeter 406, the intermediary marking area 424 or the inner perimeter 422, can indicate the expanded diameter of a tissue expander and the corresponding diameter of the implant 1110. In an example, the marking tool 400 can be used to draw the target site diameter, such as approximately 8.0 centimeters for the inner perimeter 422, approximately 9.0 centimeters for the intermediary marking area 424 or approximately 10.0 centimeters for the outer perimeter 406.

As illustrated in FIG. 14C, the medical professional then can connect the parallel lines 1412 to the diameter drawn, such as the outer perimeter 406, with extension lines 1414. As illustrated in FIG. 14D, the connection of the extension lines 1414 with, for example, the outer perimeter 406 can define a transition zone 1417 with an angle of between 100° and 120°. The transition zone can be on the region known as the tail of Spence. The transition zone 1417 can be marked. The transition zone 1417 can be a region within the patient's body where the medical professional alters or slightly alters the insertion direction of any previously discussed tool (such as the tissue dissector 600, the balloon expander 700, or the injector 800) to a direction towards the areola 1402.

The medical professional can also draw or trace along a navigation line such as the guiding line 416 extending from proximate to the incision location to the target site. The guiding line 416 can correspond to a passage extending from proximate to an incision location to the target site. Where the guiding line 416 meets or extends to the outer perimeter 406, the medical professional can draw or mark a final dissection mark 1416. The final dissection mark 1416 can indicate a final distance of insertion of the tissue dissector 600.

### Anesthesia

In an example surgical procedure, when the medical professional has completed marking the breast 1400, if desired, a cannula such as the cannula 500 described above in relation to FIG. 5 can be inserted around the area where markings have been made. In an example, the cannula 500 can be coupled via a lumen 513 with a pump, which can be connected to the source of the fluid used for anesthesia.

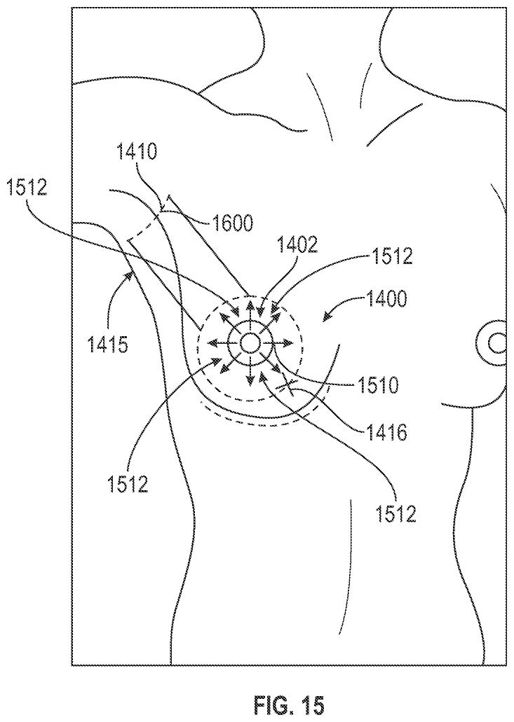

FIG. 15 illustrates an exemplary image of the areas for infiltration using a cannula in accordance with at least one example of the present disclosure. In an example, the cannula 500 can be inserted subcutaneously and into the breast tissue at several locations which could have been marked using the marking tool 400. The cannula 500 can deliver a tumescent solution into the breast tissue, via the lumen 513 attached to the pump.

The tumescent solution can be a saline solution mixed with an anesthetic. In an example, the tumescent solution can be injected into the breast 1400, the tumescent solution can numb the breast tissue to any further incisions, punctures, or forces applied in and to the breast 1400 or breast area. The saline solution can assist with saturation of the connective tissue such as the ligaments or tissue within the breast. Saturation of the ligaments and tissue can also allow stretching and expansion. In an example, approximately 500 cc of tumescent solution can be delivered to each breast with a single injection or multiple injections.

In an example as illustrated in FIG. 15, infiltration can begin on or around the incision mark 1410 which is on or around the axilla skin crease 1415 with the cannula 500. The medical professional can infiltrate the area around the incision mark 1410 with approximately 50 cc of tumescent solution. However, a different amount of tumescent solution can be delivered as dictated by the surgical procedure.

Infiltration of tumescent solution can also occur at or around the areola 1402 in a clock-like manner, as indicated by the exemplary arrows 1510 in FIG. 15. A total of approximately 350 cc of tumescent solution can be delivered around the areola 1402, or any other amount as dictated by the patient and/or the surgical procedure. Continuing infiltration can occur at or near opposing sites 1512 along, for example, the outer perimeter 406 marking, such as at an approximately 2 o'clock and 7 o'clock position or a 5 o'clock and 11 o'clock position. In an example, approximately 50 cc of tumescent solution can be delivered at each of the opposing sites 1512.

In another example, infiltration can also occur at or near the final dissection mark 1516. In an example, approximately 10 cc of tumescent solution can be delivered at or near the final dissection mark 1516.

It is also contemplated other injection points can be used and alternate amounts of tumescent solution can be delivered.

### Incision

In the example surgical procedure above, after the tumescent solution is delivered to all desired areas of the breast 1400, an incision 1600 can be made at the previously marked axillary skin crease 1415. In an example, the incision 1600 can be between approximately 1.0 centimeter and approximately 4.0 centimeters in length, such as approximately 2.5 centimeters. The incision 1600 can be cut through the dermis to the subcutaneous tissue. The incision 1600 can be approximately 3.0 millimeters to approximately 5.0 millimeters in depth. The incision 1600 can be small enough for the surgical procedure to be minimally invasive, but large enough to allow insertion of any desired or necessary tools for the surgical procedure.

### Channel Formation

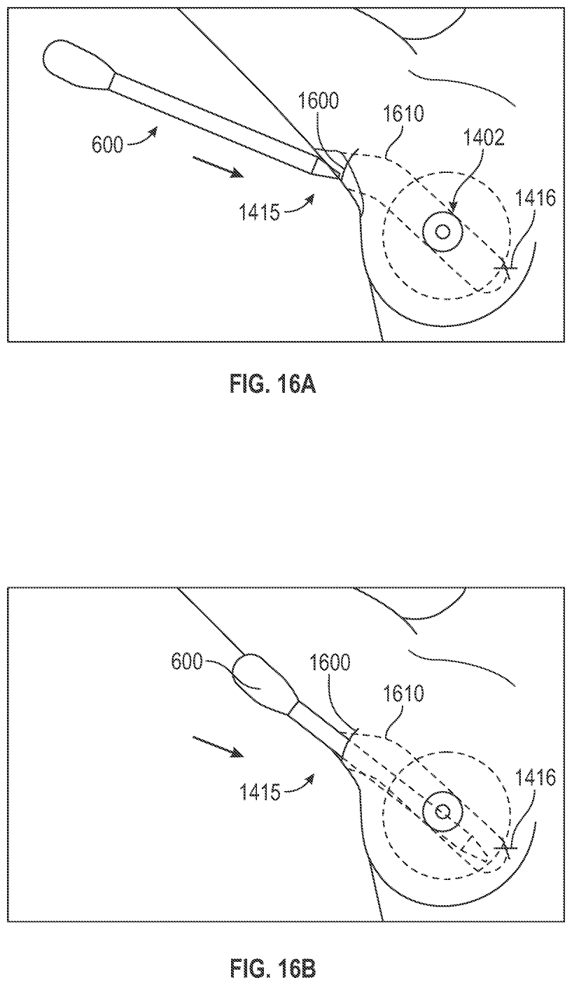

FIGS. 16A and 16B illustrate the exemplary use of a tissue dissector in accordance with at least one example of the present disclosure. Any suitable tissue dissector, such as the tissue dissector 600 described above in relation to FIG. 6, can be used for tissue dissection. In an example next step of the surgical procedure, the tissue dissector 600 can be inserted through the incision 1600, as illustrated in FIG. 16A. The tissue dissector 600 can be a reusable surgical instrument, for transient use, which can form a surgical channel by dissecting the soft tissue in an area, for example, between the axilla skin crease 1415 and the final dissection mark 1416 or other termination location.

The tissue dissector 600 can be sterilized in any known manner before insertion into the incision 1600. The tissue dissector 600 can be inserted through the incision 1600 and, for example, over the pectoralis muscle and through the breast tissue. The tissue dissector 600 can be inserted in the incision 1600 at an approximate center point of the incision 1600. The medical professional can alter or slightly alter the insertion path of the tissue dissector 600 at or near the transition zone 1417 to form a passage 1610 that can be slightly angled. At the transition point the tissue dissector 600 can be advanced over the pectoralis muscle and towards the areola 1402. The tissue dissector 600 can be advanced until it reaches the final dissection mark 1416, or the inferomedial border of the breast. In an example, the tissue dissector 600 can advance approximately 15.0 centimeters to approximately 17.0 centimeters. While inserting the tissue dissector 600, the medical professional can grasp and lift the breast 1400 to facilitate the advancement of the tissue dissector 600 which can assist in avoiding interference from the breast tissue.

The medical processional can advance the tissue dissector 600 toward the areola 1402, for example along the marked guiding line 416 (See FIG. 14C). The medical professional can keep the tissue dissector 600 at a superficial plane by pushing the tissue dissector 600 at an appropriate angle or along the contours of the patient's body. The tissue dissector 600, in this example, can form the passage 1610 within the breast tissue which can be approximately the same width as the diameter of the tissue dissector 600. The passage 1610 can be formed with the tissue dissector 600 can have a maximum width substantially similar to the dimension of the incision. In an example, when a medical professional advances the tissue dissector 600 through the incision 1600 the medical professional can strive to not damage the breast tissue or ligaments.

The medical professional can advance the tissue dissector 600 the desired distance to the target site, for example to the final dissection mark 1416. The medical professional can use the indexing markings 622 to determine the depth of the tissue dissector 600 within the passage 1610. The medical professional can then withdraw the tissue dissector 600 from the incision 1600. The medical professional can withdraw the tissue dissector 600 along or within the passage 1610 so as to not form a passage wider than the passage formed when inserting the tissue dissector or in a way which otherwise expands the passage 1610.

### Target Site Formation

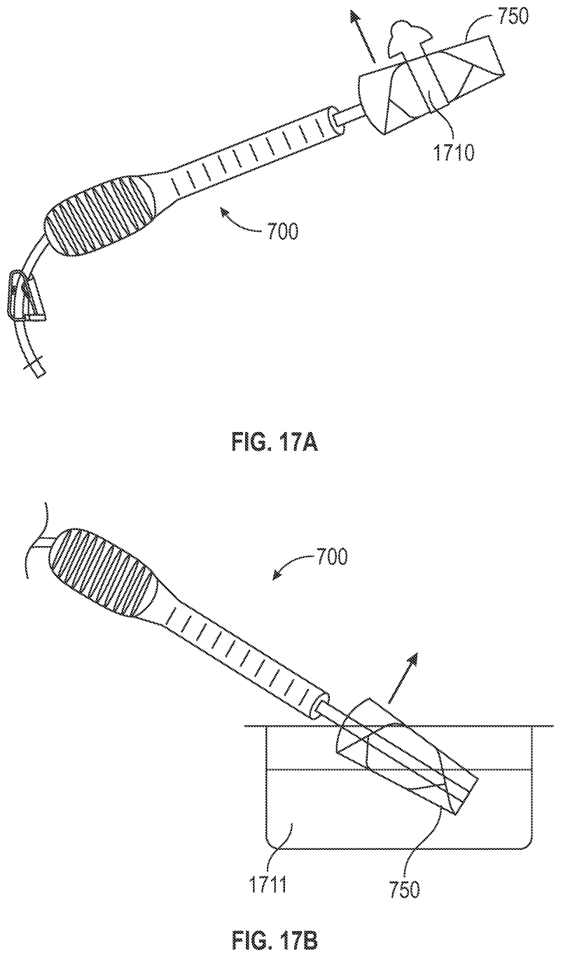

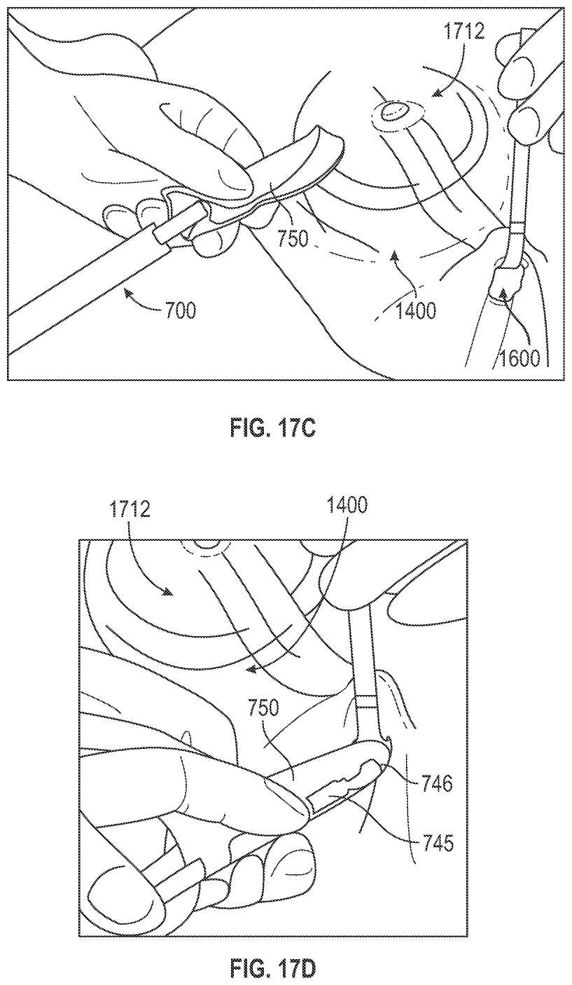

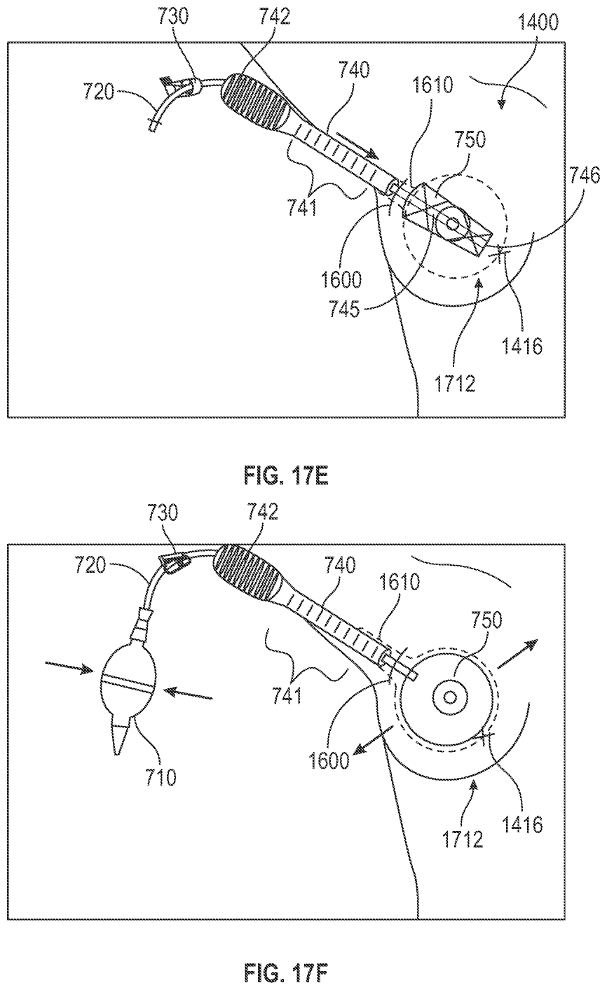

FIGS. 17A-17G illustrate exemplary images of a procedure for using an implement, such as a tissue expander in accordance with at least one example of the present disclosure. Any suitable tissued expander, such as the tissue expander 700 described above in relation to FIGS. 7A and 7B, can be used to expand the patient's tissue near the target site to create a cavity of pocket for receipt of an implant. In an example surgical procedure, the passage 1610 can be used to introduce the tissue expander 700 including the balloon 750, as discussed previously, into the target site 1712. The balloon 750 can be supplied to a medical professional wrapped, rolled or otherwise compressed and bound with a removable holding device 1710 such as a tie, strap, tape, clip or the like when supplied by the manufacturer, as illustrated in FIG. 17A. The balloon 750 can be reduced in size when supplied such that the balloon 750 can be inserted to minimize any unwanted interaction with previously inserted tools or anatomy. In an example, the removable holding device 1710 can be removed from the balloon 750.

The medical professional can then subject the balloon 750 to a hydration solution 1711, as illustrated in FIG. 17B, thereby hydrating the balloon 750. For example, the balloon 750 can be hydrated in a sterile saline solution.

In an example surgical procedure, after hydrating the balloon 750, the medical professional can refold, reroll or otherwise compress the balloon 750 to a size or profile to fit within the incision 1600, as illustrated in FIG. 17C.

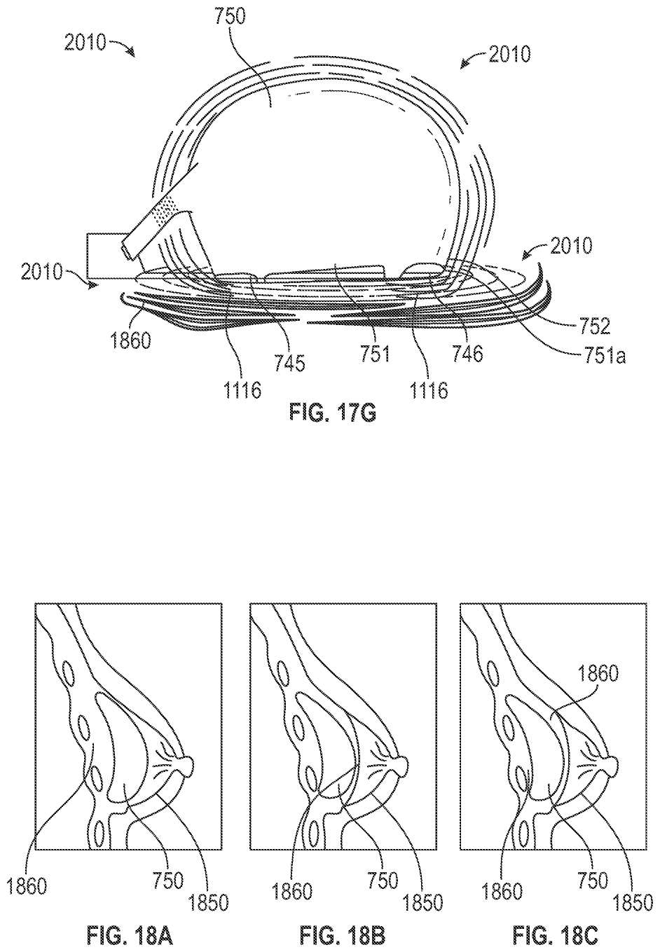

FIGS. 18A-18C illustrate side profile views depicting exemplary balloon placement locations within the target site in accordance with various examples of the present disclosure. As illustrated in FIG. 18A, in one example the balloon 750 can be advanced subglandularly, or under the mammary tissue 1850 but over the fascia layer surrounding the pectoralis muscle 1860. In another example, as illustrated in FIG. 18B, the balloon 750 can be advanced subpectroally, or under the pectoralis muscle 1860. In another example, as illustrated in FIG. 18C, the balloon 750 can be inserted dual planarly, or partly under both the pectoralis muscle 1860 and the mammary tissue 1850. The location of implantation can be at the medical professional's discretion and based upon the anatomical structure of the patient

Turning back to FIGS. 17A-17G, and in particular FIG. 17D, the medical professional can insert the folded, rolled or otherwise compressed balloon 750 through the incision 1600. The balloon 750 can be inserted with the supporting extension 745 oriented as the posterior base of the balloon 750. The supporting extension 745 can assist in advancing the balloon 750 through the passage 1610. In an example, the tip 746 of the supporting extension 745 can be used to advance the balloon 750 if any breast tissue inhibits advancing the balloon 750 to the target site 1712.

As illustrated in FIG. 17E, the medical professional can advance the balloon 750 until the tip 746 of the supporting extension 745 reaches the final dissection mark 1416 and the target site. The medical professional can also use the indexing markings 741 on the introducer 740 to determine or locate how far the balloon 750 has been inserted.

A pump, such as the manual pump 710, can be used to inflate or expand the balloon 750. The manual pump 710 can be coupled to the flexible lumen 720 such that the flexible lumen 720 extends from the manual pump 710 to the balloon 750. As illustrated in FIG. 17F, the medical professional can then activate the pump 710 by, for example, squeezing the pump 710 until the balloon 750 reaches a desired expanded or inflated state, such as fully inflated. In an example, the medical professional can squeeze the pump between approximately 3 and approximately 12 times, such as approximately 4 or approximately 5 times, for the balloon 750 to reach its fully expanded, or desired, state. In an example, the fully expanded balloon 750 can have a base width or diameter between approximately 10 centimeters to approximately 20 centimeters, or between approximately 12 centimeters and 16 centimeters, or for example approximately 14 centimeters. Other suitable pumps, such as an electric pump, can be used in place of the manual pump.

The inflated balloon 750 can push or apply a force laterally against connective tissue such as soft tissue or ligaments 2010 (hereinafter “connective tissues” can refer to either ligaments or tissue) within the breast 1400, as illustrated in FIG. 17G. The connective tissues 2010 can be the Cooper ligaments which can shape and support the breast. In an example, when the balloon 750 inflates it can project away from the supporting extension 745, or project anteriorly. By projecting anteriorly, the balloon 750 can assist in the formation of a cavity 1750 which can maintain the anatomically original connective tissue structure and musculature at the posterior side 751 of the balloon 750. During expansion of the balloon 750, the connective tissues 2010 can be minimally separated around rounded edges 752 of the posterior side 751 of the balloon 750. When the balloon 750 expands, the balloon 750 can preserve at least one point of attachment 1116 of the connective tissues 2010. The at least one point of attachment 1116 can be located laterally of expanded balloon 750. The point of attachment 1116 can also be located inferiorly of the expanded balloon 750. This point of attachment 1116 can be located adjacent to the curved, posterior side 751 of the balloon 750, such that the ligament point of attachment is posteriorly formed relative to the expanded balloon.

During expansion of the balloon 750, the passage 1610 can remain substantially intact or remain substantially maintained. Expansion of the balloon 750 can form a cavity 1750 only around the balloon 750 and which does not expand the passage 1610 through which the balloon 750 traversed to reach the target site. The dimension of the incision 1314 can also be substantially maintained as originally formed.

In an example, the balloon 750 can expand and project anteriorly, thereby separating the connective tissues 2010 as it advances away from, for example, the pectoralis muscle 1860. The balloon 750 can expand, for example, against the connective tissues 2010 such that the connective tissues 2010 can surround, encase, hug or nest around the balloon 750. The connective tissues 2010 surrounding the balloon 750, as illustrated in FIG. 17G, can form a nest-like cavity 1750 to hold the balloon 750 such that the balloon 750 can apply a force laterally, medially and/or inferiorly against the connective tissues 2010. In an example, the balloon 750 can push the connective tissues 2010 laterally. The balloon 750 can separate the connective tissues 2010 in a way to preserve the connective tissue and minimally damage the internal structure of the breast 1400. In an example, the structure of the separated connective tissues 2010 can maintain a nearly original anatomical structure.

The medical professional can then close the clip 730 when the balloon 750 has been expanded by the desired amount. In an example, the clip 730 can be closed to keep the balloon 750 inflated for predetermined amount of time. In an example, the balloon the predetermined amount of time the balloon can remain inflated for between approximately 3 and approximately 7 minutes, such as approximately 5 minutes. Maintaining the balloon 750 in the inflated state can allow the balloon to act as a hemostatic implement and can assist in stopping or minimizing any bleeding which can occur within the breast area. The inflated balloon 750 can be slightly over-inflated to apply pressure to the surrounding tissues and ligaments which can assist in ceasing or minimizing any bleeding. The balloon 750 can be held in an inflated, or expanded, state for a desired amount of time. In an example when the balloon 750 is held in an inflated state for a desired amount of time, the medical professional can repeat the steps discussed above for the patient's other breast. The medical professional can open the clip 730 to deflate the balloon 750 after the balloon 750 is held for the desired amount of time. The deflated balloon 750 can then be withdrawn from the patient's breast.

The balloon 750 can be withdrawn through the passage 1610. In an example, the balloon 750 can traverse the passage 1610 without increasing, or only minimally increasing, the width or the diameter of the passage 1610. In an example, the balloon 750 can be withdrawn and removed through the incision 1600 while maintaining (or at least substantially maintaining) the initial size of the incision 1600

In an example, the balloon 750 can be sized, shaped, or otherwise structured in a manner that mimics or is at least substantially similar to the implant 1110 that will be inserted such that the resulting cavity 1750 is sized to hug or nest the implant 1110 after insertion. The balloon 750 can be sized similar to the implant that will be inserted. The balloon 750 can be sized such that the balloon 750 can form a cavity that is minimally smaller than the desired implant. In an example, it is not preferred for the balloon 750 to be significantly larger than the desired implant. In an example, it is not preferred for the balloon 750 to be more than minimally over-inflated such that the balloon 750 can form a cavity larger than the desired implant. Minimizing the amount of “excess” space formed in the cavity 1750 can prevent or minimize movement of the implant 1110 after insertion. In an example, the balloon 750 can be sized, shaped, or otherwise structured such that the resulting cavity 1750 is slightly undersized compared to the implant 1110 to ensure secure placement and positioning of the implant 1110 in the cavity 1750.

### Implant Insertion

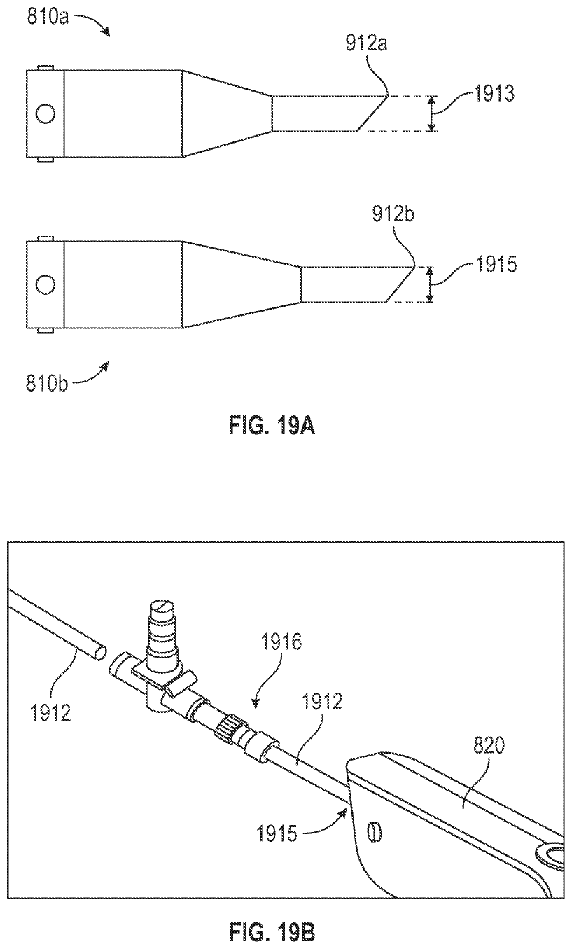

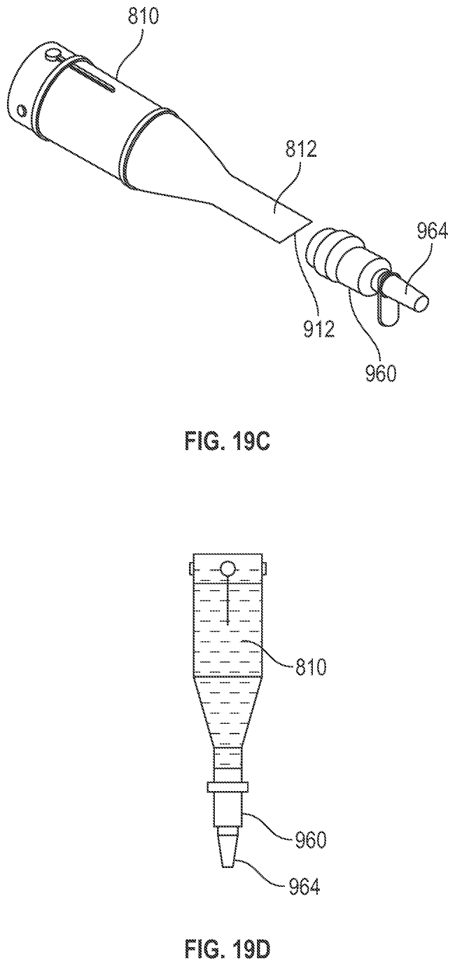

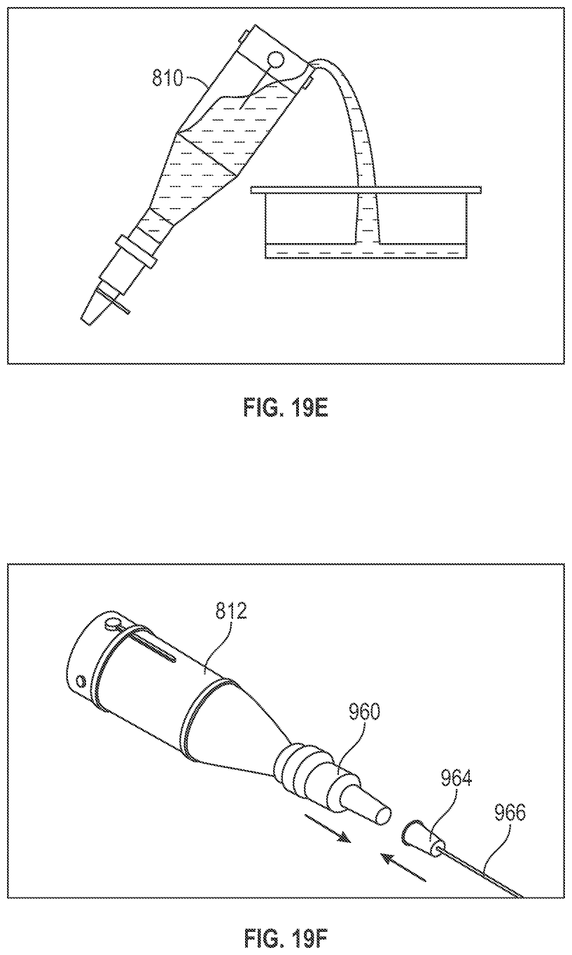

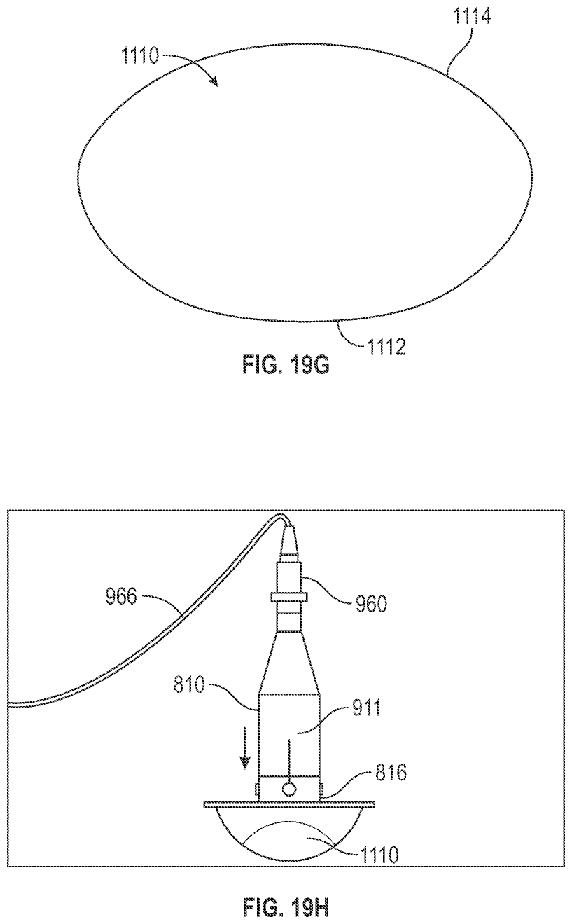

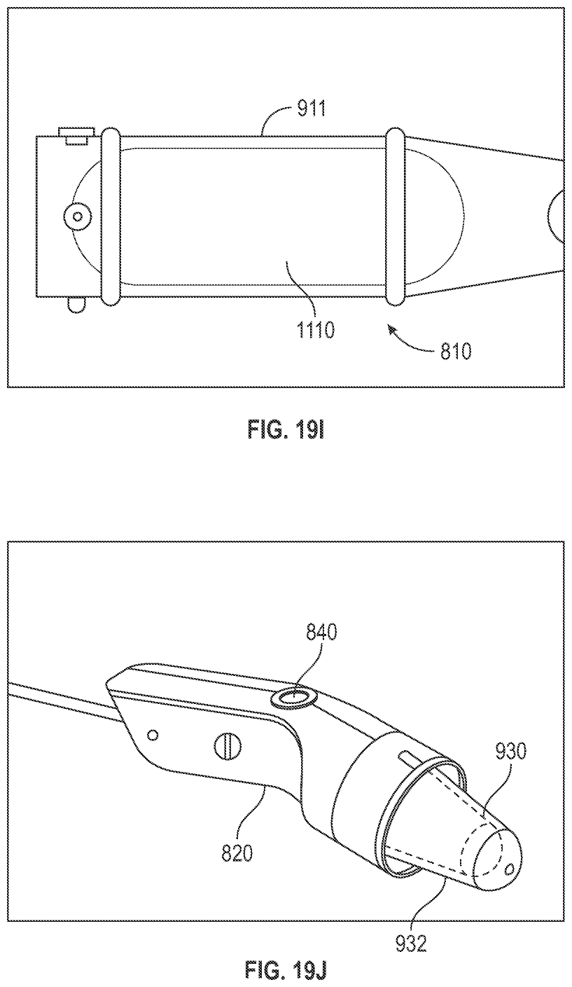

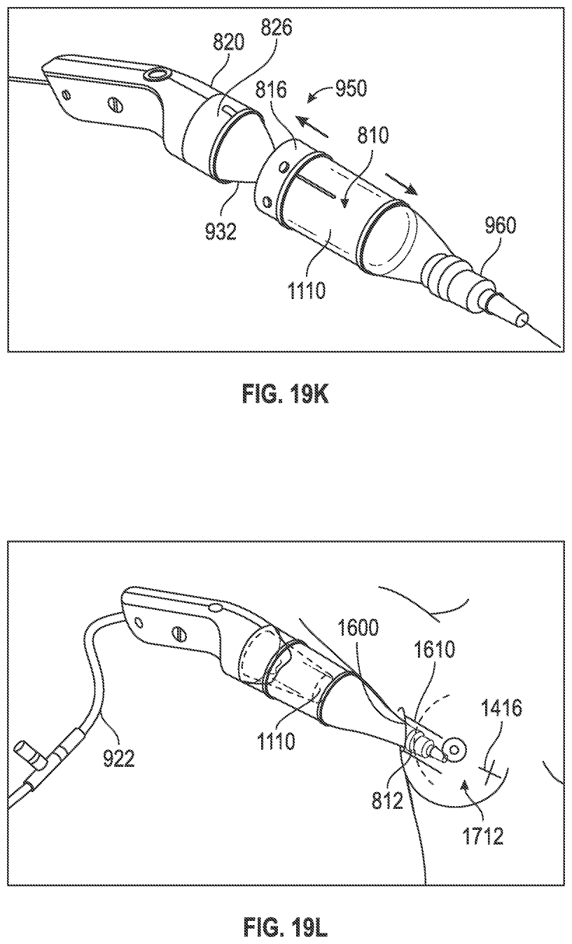

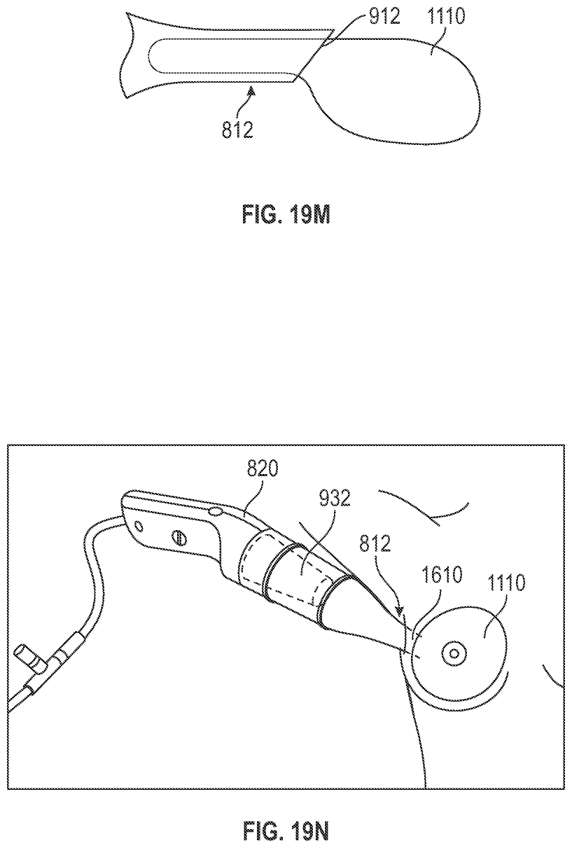

FIGS. 19A-19N illustrate exemplary images of a procedure for inserting an implant into a target site in a breast in accordance with at least one example of the present disclosure. In an example, the injector 800 with the nozzle end opening 912 can be used to insert the implant 1110 into the cavity 1750 formed by the balloon 750.

As illustrated in FIG. 19A, the medical professional can select the appropriately sized nozzle 810 for housing and then expelling the implant 1110. With reference to FIG. 19A, the medical professional can select between a nozzle 810a with a nozzle end opening 912a having a first diameter 1913 and a nozzle 810b with a nozzle end opening 912b having a second diameter 1915. In an example, the first diameter 1913 can be between approximately 15 millimeters and approximately 25 millimeters and the second diameter 1915 can be between approximately 20 millimeters and approximately 32 millimeters. In an example, the first diameter 1913 can be approximately 20 millimeters and the second diameter 1915 can be approximately 23 millimeters. However, any nozzle 810 with any suitably-sized nozzle end opening 912 can be used.

The medical professional can utilize an external air-pressure system to both draw the implant 1110 into the nozzle 810 and to subsequently expel the implant 1110 from the nozzle 810. In an example, a separate air-pressure system can be used to draw the implant 1110 in and another air-pressure system to expel the implant 1110 from the nozzle 810. In an example, the external air-pressure system can be calibrated to between approximately 4.8 to approximately 5.6 bar (approximately 70 to approximately 80 psi).

The implant 1110 can have a first configuration or neutral configuration, before the implant 1110 is drawn into the nozzle 810. The first configuration can be one in which each of the anterior side and the posterior side projects away from the longitudinal axis. The implant 1110 can then have a second configuration when the implant 1110 can be contained within the housing of the nozzle 810 such that the anterior side and the posterior side can be compressed towards the longitudinal axis. The implant 1110 can have an implanted configuration when the implant has been expelled from the nozzle 810 such that the posterior side is compressed against the anatomical structure of the patient and the anterior side of the implant projects further from the longitudinal axis than when in the first, or neutral, configuration.

As illustrated in FIG. 19B, the medical professional can couple the pressure regulator 860 to the external air-pressure system through a medical grade tubing 1912. In an example, the medical grade tubing 1912 can have an outer diameter of approximately 6.35 millimeters (approximately 0.25 inches). The distal end 1914 of the medical grade tubing 1912 can be connected with the proximal end of the handle 820 and the proximal end 1916 of the medical grade tubing 1912 can be connected with the pressure regulator 860.

As illustrated in FIG. 19C, the vacuum cup 960 with the vacuum cap 964 can be coupled to the nozzle end 812. The vacuum cup 960 and the vacuum cap 964 can cover and seal the nozzle end opening 912. However, other means of sealing the nozzle end 812 can be used as the device and surgical procedure dictate.

As illustrated in FIG. 19D, the nozzle 810 can, for example, be held with the nozzle end 812 facing downward with the vacuum cup 960 facing downward. The medical professional can then fill the nozzle 810, at least partially, with a sterilizing solution, such as a sterile saline solution. In an example, an internal coating on an internal surface 811 of the nozzle 810 can be activated when the internal coating comes in contact with the sterilizing solution. In another example, the internal surface 811 of the nozzle 810 does not have a coating. In another example, the internal coating can be activated with a different liquid solution, or without a liquid solution.

As illustrated in FIG. 19E, the nozzle 810 can then be emptied after holding the sterilizing solution for a predetermined amount of time, such as a few seconds or minutes after the sterilizing solution has been used.

As illustrated in FIG. 19F, the vacuum cap 964 can be removed from the vacuum cup 960. The vacuum cup 960 can remain on the nozzle end 812. The vacuum cup 960 can then be attached to the external air-pressure system, or another similar system. For example, the external air-pressure system can be coupled with the vacuum cup 960, through a sterile suction tubing 966. The sterile suction tubing 966 can have an outer diameter between approximately 4.75 millimeter to approximately 6.4 millimeters, or any diameter dictated by the purpose.

The implant 1110, as illustrated in FIG. 19G, can be a compressible implant such that when subjected to external pressures, the implant 1110 can expand in a lateral direction, approximately equivalent in any expansion direction. In an example as illustrated in FIG. 19G, when the implant 1110 is resting on a surface, the posterior side 1112, or the side resting on or against a surface, can flatten and the opposing side, or anterior side 1114, can project in a direction away from the surface, or project anteriorly.

The implant 1110 can then be hydrated, as illustrated in FIG. 19H in a saline solution or any other suitable solution. The saline solution can aid in the implant 1110 being expelled from the nozzle 810, through the nozzle end 812 and the nozzle end opening 912. The implant 1110 can be hydrated in a container from which the implant 1110 can be removed without being touched by a non-sterile source, such as a hand or non-sterile medical tool.

As illustrated in FIG. 19H, the nozzle 810 can be connected to the external air-vacuum system via the sterile suction tubing 966. The medical professional can place the nozzle 810 such that the nozzle engagement area 816 can face downward and opened toward the implant 1110 in the container used to hydrate the implant 1110. In an example, the medical professional can activate, or turn on, the external air-vacuum system such that a low-pressure environment can be formed within the nozzle 810. The low-pressure environment, or vacuum, can draw the implant 1110 into the nozzle 810 when the nozzle 810 can be placed proximate to the implant 1110. A vacuum pressure of between approximately 508.0 to approximately 762.0mmHg (approximately 20.0 to approximately 30.0 inHg) can be supplied through a lumen to the vacuum opening 962 and the vacuum cup 960. In another example, when the nozzle 810 can be placed above the implant 1110, the low-pressure environment within the nozzle can draw the implant 1110 inside. In an example, the implant 1110 can be substantially uniformly compressed and elongated when drawn into the middle portion 911 of the nozzle 810, as illustrated in FIG. 19I. In an example the implant 1110 can be compressed in a longitudinal and vertical direction when within the middle portion 911 of the nozzle 810. The implant 1110 can be compressed such that the posterior and anterior sides are closer to each other, and the top and the bottom sides are closer to each other. The implant 100, for example, can be compressed substantially equally about the longitudinal axis 151 and the vertical axis 130.

The medical professional can, in an example, verify the expansion of the expandable membrane 932 as illustrated in FIG. 19J. The medical professional can first activate, or turn on, the external-air pressure system in a manner that a specified external-air pressure from the external air-pressure system can be communicated through the handle 820. The medical professional can then depress or otherwise engage the actuator 840, such as via a push-button or any way which can control the expansion of the expandable membrane 932. In an example, activating the actuator 840, such as depressing a button, can cause pressurized air, or any suitable fluid, to enter the expandable membrane chamber 930 coupled to the handle 820. When the actuator 840 is depressed, it can cause the fluid supply lumen 922 to meet, couple or connect with the fluid supply mouth 924. The pressurized air can then enter the expandable membrane chamber 930 through the fluid supply mouth 924 and into the expandable membrane 932. The expandable membrane 932 can then expand. The medical professional can release the actuator 840 which can cause the pressurized air to exit the expandable membrane chamber 930 through the use of the vent switch 842. When the pressurized air exits the expandable membrane chamber 930, the expandable membrane 932 can deflate. In another example, the actuator 840 can be held in a depressed, or otherwise held in place, to maintain expansion of the expandable membrane 932. When the actuator 840 is released, the pressurized air in the expandable membrane chamber 930 can be released through the fluid supply mouth 924. The expandable membrane 932 can then return to substantially its original position and shape.

The nozzle 810 with the implant 1110 contained within the middle portion 911 can be coupled with the handle 820, as illustrated in FIG. 19K. In an example, the attachment mechanism 950 on the handle 820 and the nozzle 810 can be aligned. The nozzle engagement area 816 can couple with the handle engagement area 826 using the attachment mechanism 950. In an example, there can be an audible notification, such as a click, when the nozzle engagement area 816 completes coupling with the handle engagement area 826. The audible notification can indicate the nozzle 810 and the handle 820 are substantially or completely sealed. The attachment mechanism 950 can offer a visual indication, such as appropriate alignment, for example with an “L” shaped engagement.

The vacuum cup 960 can be detached from the nozzle end 812 when the handle 820 and the nozzle 810 are coupled. The medical professional can then prepare to insert the implant 1110 into the target site 1712 after removing the vacuum cup 960.

The medical professional can insert the nozzle end 812 through the incision 1600, as illustrated in FIG. 19L. The medical professional can then advance the nozzle end 812 through the passage 1610 until the nozzle end 812 reaches the target site 1712. When the medical professional advances the nozzle end 812 through the passage 1610, the medical professional can strive to avoid increasing the width or dimension of the passage 1610. The medical professional can use the markings, such as parallel lines 1412 and the guiding line 416, to guide the nozzle end 812 to the target site 1712.

When the nozzle end 812 reaches the target site 1712 the medical professional can depress or engage the actuator 840. The actuator 840 can cause the fluid supply lumen 922 to connect, meet or otherwise couple with the fluid supply mouth 924. The connecting, meeting or other coupling the fluid supply lumen 922 with the fluid supply mouth 924 can provide a pathway for the pressurized fluid to pass into the expandable membrane 932. The expandable membrane 932 can then be expanded or inflated. The expandable membrane 932 can be filled with pressurized fluid rapidly and therefore the expandable membrane 932 can be expanded at such a rate the expandable membrane 932 applies a pressure or force against the implant 1110 within the middle portion 911. The pressure or force from the expandable membrane 932 can force the implant 1110 toward the nozzle end opening 912.

The expandable membrane 932 can be expanded by an amount that is sufficient to force the expandable membrane 932 against the implant 1110. This force can cause the implant 1110 to further compress as the implant 1110 passes into the nozzle end 812, as illustrated in FIGS. 19M and 19N. The implant 1110 can then further compress within the nozzle end 812 and the force from the expandable membrane 932 which can be applied against the implant 1110 can expel the implant 1110 from the nozzle end opening 912 and into the target site 1712, as illustrated in FIG. 19N. The delivery pressure can be regulated to approximately 3.0 bar to approximately 4.5 bar (approximately 50 psi to approximately 60 psi). The delivery pressure can be higher than the vacuum pressure previously described.

The nozzle end opening 912, in an example, can have an angled, tapered, or otherwise shaped opening, for example as illustrated in the examples shown in FIGS. 12A-D. The size and shape of the nozzle end opening 912 can provide a length to more closely place the implant 1110 in the target site. The size and shape of the nozzle end opening 912 can allow stresses to be reduced against the implant 1110 in such a way as to not damage the implant 1110. The size and shape of the nozzle end opening 912 can allow the implant 1110 to more gradually expand or otherwise enter the target site 1712 without damage, or with minimal damage. As the implant 1110 is released from the nozzle end 812 through the nozzle opening 912, the implant 1110 can expand to a substantially original form. In an example, the nozzle end 812 combined with the geometry of nozzle end opening 912 can draw the implant 1110 out of the nozzle end 812. The implant 1110 can expand through the angular, or larger, nozzle end opening and then can be released from the nozzle end opening 912. The angled nozzle end opening 912 can allow the stresses and forces applied to the implant 1110 to be applied gradually such that the implant 1110 can be implanted with minimal, if any, damage to the implant 1110. The angled nozzle end opening 912 can result in the implant 1110 expanding to its original form gradually. The stresses or forces applied to the implant can be reduced against different surfaces of the implant 1110 at different times.

The implant 1110 can be symmetrical about a longitudinal axis. In an example with a symmetrical implant, such as implant 100, the posterior side can be the anterior side and visa versa. The implant 100 can be inserted in any orientation relative to the posterior side and the anterior side.

In an example, when the implant 1110 is expelled from the nozzle end 812, the actuator 840 can immediately, or shortly thereafter, be released and the expandable membrane 932 can thereby be deflated. The medical professional can then remove the nozzle end 812 from the incision 1600 along the same passage 1610 as formed by the tissue dissector 600. The medical professional can then close the incision 1600 with sutures or any known method of closing an incision.

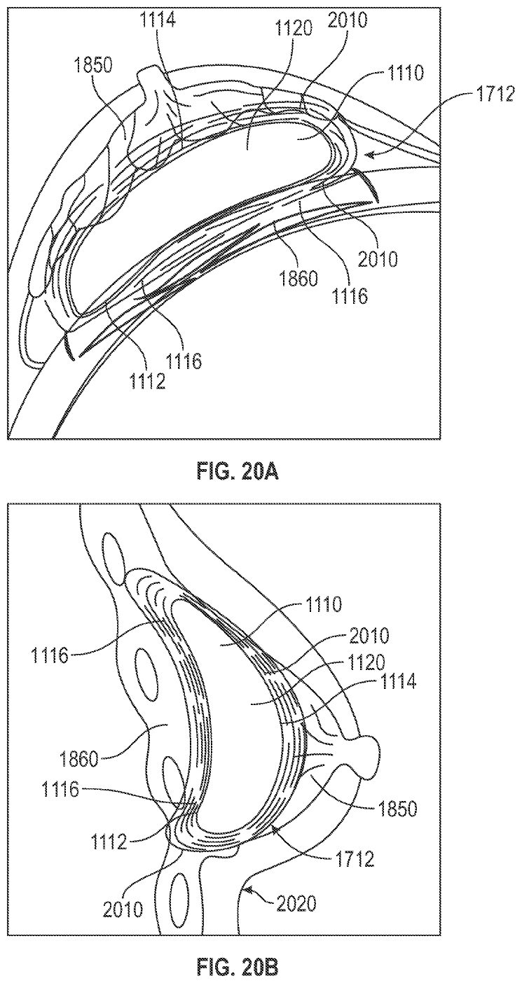

FIG. 20A illustrates a cross sectional view of a breast with an implant inserted in a reclined orientation in accordance with at least one example of the present disclosure. FIG. 20B illustrates a cross sectional view of a breast with an implant inserted in an upright orientation in accordance with at least one example of the present disclosure. As illustrated in FIG. 20A and FIG. 20B, the implant can be inserted into the target site 1712 according to the previously discussed procedure. The implant 1110 can then be stabilized by the connective tissues 2010 surrounding the implant 1110. In an example, the implant 1110 can be nested within the target site 1712 such that the ligaments can hug, surround or otherwise hold the implant 1110. In an example, the connective tissues 2010 (which were carefully pushed aside and left intact when inflating the balloon 750) can wrap around or support the implant 1110. The connective tissues 2010 can wrap around or support portions of the implant 1110 on all sides including the posterior side 1112 of the implant 1110. The connective tissues 2010 can curve under at least one of the top, bottom, right and left sides of the implant 1110 and under the posterior side 1112. The connective tissues 2010 can curve under the implant 1110. The connective tissues 2010 can be wrapped around the implant 1110 to be in ligament-to-surface contact with substantially all surfaces of the implant 1110. The posterior side 1112 of the implant 1110 which can be supported by the connective tissues 2010 wrapped underneath the posterior side can provide a greater projection of the anterior side 1114 of the implant 1110. The connective tissues 2010 can provide a nesting or cupped support of the posterior side 1112 of the implant.

The connective tissues 2010 can provide at least at least one point of attachment 1116 which can support the implant 1110. The point of attachment 1116 can be the point of the connective tissues 2010 where the connective tissues 2010 are not separated but can remain attached as in the original anatomical form. The point of attachment 1116 can be located laterally of the implant 1110 such that the connective tissues 2010 can assist in resisting lateral movement of the implant 1110. The point of attachment 1116 can be located inferiorly of the implant to assist in resisting inferior movement of the implant 1110.

As illustrated in FIGS. 20A and 20B, the implant 1110 can be located in the target site 1712 and can have a form where the posterior side 1112 can be flatter than the anterior side 1114. In this example, the filler 1120 within the implant 1110 can cause the shell of the implant 1110 to form in a substantially D-like shape where the posterior side 1112 can conform to the pectoralis muscle and the anterior side 1114 can project forward and away from the pectoralis muscle 1860. In an example, the connective tissues 2010 at the target site 1712 can preserve the substantially D-like shape of the implant 1110. The connective tissues 2010 can act to minimize lateral and downward movement of the implant 1110 due substantially to the nesting effect of the ligaments holding the implant 1110 in place.

In an example, the implant 1110 can be inserted into a higher area of the breast than implants known in the art. In an example, the target site 1712 can be aligned more closely with the upper regions of the breast than target sites previously known in the art. The posterior side 1112 of the implant 1110 can rest against the pectoralis muscle 1860, such as the pectoralis muscle wall, which can cause the anterior side 1114 to project into the breast tissue 1850. In the example illustrated in FIGS. 20A and 20B, the implant 1110 can remain above the inframammary fold 2020. The alignment of the implant 1110 within the target site 1712 can provide the patient with a body structure similar to before the implant 1110 was inserted. In an example where the implant 1110 can be inserted in the above-described method, the implant 1110 can remain substantially stationary and not slide or move laterally when the patient reclines.

Implants such as the implant 1110 that have been surgically implanted into a patient using the procedure described herein have shown a greater projection of the implant and/or the breast than traditional flat-backed implants that have been surgically implanted into a patient using previously known methods of breast implantation. For example, the present inventors have found that implants (such as the implant 1110) inserted by the above-described method can project approximately 10 to 40 percent further than flat-backed implants inserted by previously known breast implantation methods. In other examples, the above-described method of implantation can provide an increased projection between 20 and 30 percent, such as 22 percent, as compared to implants inserted by previously known breast implantation methods.

### Various Notes and Aspects

Aspect 1 can include an implant insertion system comprising a tissue expander including an inflatable balloon, the balloon having a curved posterior side when inflated and an implant having a curved posterior side when positioned against an anatomical surface, wherein the curved posterior side of the implant substantially matches the curved posterior side of the tissue expander.

Aspect 2 can include, or can optionally be combined with the subject matter of Aspect 1, to optionally include an anterior side; a longitudinal axis positioned between the anterior side and the curved posterior side; and a posterior apex disposed on the curved posterior side, the posterior apex extending further from the longitudinal axis in a first configuration than when the implant is positioned against the anatomical surface.

Aspect 3 can include, or can optionally be combined with the subject matter of Aspect 1 or Aspect 2, to optionally include the anterior side of the implant is curved in a manner substantially similar to the curved posterior side, the anterior side of the implant includes an anterior apex, and the longitudinal axis is positioned substantially equidistant from the anterior apex and the posterior apex.

Aspect 4 can include, or can optionally be combined with the subject matter of any of Aspects 1-3, to optionally include implant further including an anterior side having a curved profile with an apex substantially similar to a curved anterior side of the balloon.

Aspect 5 can include, or can optionally be combined with the subject matter of any of Aspects 1-4, the balloon has a compressed configuration and an inflated configuration.

Aspect 6 can include, or can optionally be combined with the subject matter of any of Aspects 1-5, wherein when the balloon is in the compressed configuration the balloon is sized to fit through a small incision.

Aspect 7 can include, or can optionally be combined with the subject matter of any of Aspects 1-6, wherein the balloon is configured to have a substantially similar diameter in the compressed configuration and the inflated configuration.

Aspect 8 can include a method of forming a nest-like cavity within a breast area of a patient's body comprising: making an incision having a specified dimension at a desired location of the patient's body; inserting a tissue expander having a balloon into a target site of the patient's body; inflating the balloon at the target site between at least two layers of connective tissue to separate the at least two layers of connective tissue and form a cavity with a profile substantially matching a profile of a breast implant. Where an anterior side of the balloon has a curved profile and a posterior side has a curved profile. Where as the balloon is inflated, the balloon is projected in an anterior direction and a posterior direction. Where when the balloon is substantially fully inflated, the at least two layers of connective tissue surround the balloon. The method can include maintaining the balloon in an inflated state; deflating the balloon after a predetermined period of time; and removing the tissue expander and the balloon from the patient's body.

Aspect 9 can include, or can optionally be combined with the subject matter of Aspect 8, to optionally include where the balloon is inflated to expand anteriorly to separate the connective tissue.

Aspect 10 can include, or can optionally be combined with the subject matter of any of Aspect 8 or Aspect 9, to optionally include where the at least two layers of connective tissue are at least one of ligaments and soft tissue; and where when the at least one of ligaments and soft tissue are minimally separated around the posterior side of the balloon.

Aspect 11 can include, or can optionally be combined with the subject matter of any of Aspect 8-10, to optionally include the at least two layers of ligaments surround, encase, hug or nest the balloon.

Aspect 12 can include, or can optionally be combined with the subject matter of any of Aspect 8-10, to optionally include wherein after separation, the at least two layers of ligaments maintain a nearly original anatomical structure.

Aspect 13 can include an implant insertion method comprising: expanding a tissue expander to form a target site in a patient's body, the tissue expander having a curved posterior side that preserves at least one ligament point of attachment posteriorly of the tissue expander; and inserting an implant into the target site in the patient's body, the implant having a curved posterior side supported by the at least one ligament point of attachment posteriorly of the implant.

Aspect 14 can include, or can optionally be combined with the subject matter of Aspect 13, to optionally include wherein the at least one ligament point of attachment is located: laterally of a center point of the implant to resist lateral movement of the implant; or inferiorly of the implant to resist inferior movement of the implant.

Aspect 15 can include, or can optionally be combined with the subject matter of any of Aspect 13 or Aspect 14, to optionally include wherein the tissue expander includes a balloon having a compressed configuration and an expanded configuration.

Aspect 16 can include, or can optionally be combined with the subject matter of any of Aspect 13-15, to optionally include expanding the tissue expander to separate at least two layers of connective tissue at the target site to form a cavity while maintaining the at least one ligament point of attachment posteriorly of the tissue expander; wherein the tissue expander comprises a balloon that projects in a posterior direction and in an anterior direction to separate the at least two layers of ligaments.

Aspect 17 can include, or can optionally be combined with the subject matter of any of Aspect 13-16, to optionally include wherein the at least two layers of connective tissue further includes ligaments; wherein the ligaments wrap around and support the implant when the implant is inserted into the target site.

Aspect 18 can include, or can optionally be combined with the subject matter of any of Aspect 13-17, to optionally include making a small incision in a desired area of the patient's body; inserting the tissue expander through the small incision; and inserting the implant through the small incision.

Aspect 19 can include, or can optionally be combined with the subject matter of any of Aspect 13-18, to optionally include wherein the implant is inserted into the target site with an injector.

Aspect 20 can include, or can optionally be combined with the subject matter of any of Aspect 13-19, to optionally include wherein the target site is in a patient's breast area.

Aspect 21 can include, or can optionally be combined with the subject matter of any of Aspect 13-20, to optionally include wherein the tissue expander, when in an expanded state, is sized and shaped similar to the implant.

Aspect 22 can include, or can optionally be combined with the subject matter of any of Aspect 13-21, to optionally include wherein inserting the implant into the target site comprises loading the implant into an injector having a nozzle with a cavity, a nozzle end and a nozzle end opening, wherein the implant is compressed within the cavity of the nozzle between the nozzle end and an expandable membrane; engaging an actuator on the injector coupled with a pressurized fluid source and coupled with the expandable membrane, where the fluid source provides pressurized fluid into the expandable membrane. The method can further include applying a force to the implant with the expandable membrane; expelling the implant through the nozzle end and out of the nozzle end opening and into the target site; and maintaining the at least one ligament point of attachment during and after expelling the implant into the target site.

Aspect 23 can include, an implant insertion system comprising: an injector including a nozzle that defines a housing; and an implant including an anterior side, a posterior side, and a longitudinal axis between the anterior side and the posterior side, the implant having: a first configuration in which each of the anterior side and the posterior side projects away from the longitudinal axis, wherein the first configuration comprises a neutral configuration; a second configuration in which each of the anterior side and the posterior side is compressed towards the longitudinal axis when contained within the housing of the nozzle; and an implanted configuration in which the posterior side is compressed against an anatomical structure and the anterior side projects further from the longitudinal axis than when in the first configuration.

Aspect 24 can include, or can optionally be combined with the subject matter of Aspect 23, wherein the anterior side and posterior side of the implant each projects a substantially similar distance away from the longitudinal axis in the first configuration.

Aspect 25 can include, or can optionally be combined with the subject matter of any of Aspect 23 or Aspect 24, to optionally include wherein the anterior side of the implant projects approximately 10 percent to approximately 40 percent further in the implanted configuration than in the first configuration.

Aspect 26 can include, or can optionally be combined with the subject matter of any of Aspects 23-25, to optionally include wherein the anterior side of the implant projects approximately 20 percent to approximately 30 percent further in the implanted configuration than in the first configuration.

Aspect 27 can include, or can optionally be combined with the subject matter of any of Aspects 23-26, to optionally include wherein the anterior side and the posterior side are substantially uniformly compressed in the second configuration.

Aspect 28 can include, or can optionally be combined with the subject matter of any of Aspects 23-27, to optionally include wherein the implant is a breast implant

Aspect 29 can include, or can optionally be combined with the subject matter of any of Aspects 23-28, to optionally include wherein the implant is symmetrical about at least two axes.

Aspect 30 can include, or can optionally be combined with the subject matter of any of Aspects 23-29, to optionally include wherein the injector further includes: a handle detachably coupled with the nozzle. The handle includes an expandable membrane configured to expand into the nozzle with a supply of a pressurized fluid. The handle includes an actuator to open and close a connection between a pressurized fluid source and the expandable membrane.

Aspect 31 can include a method of inserting an implant having an anterior side, a posterior side, and a longitudinal axis between the anterior side and the posterior side into a patient's body comprising: drawing the implant into a nozzle cavity of an injector nozzle to transform the implant from a first configuration in which both the anterior and posterior sides of the implant project away from the longitudinal axis to a second configuration in which both the anterior and posterior sides of the implant are compressed towards the longitudinal axis; coupling a handle having an expandable membrane with the injector nozzle such that the implant is disposed between a nozzle end opening and the expandable membrane; applying a force with the expandable membrane against a proximal end of the implant; and expelling the implant through the nozzle end opening and into a target site. Where expelling the implant into the target site allows the implant to expand to an implanted configuration in which the posterior side is compressed against an anatomical structure and the anterior side projects further from the longitudinal axis than when in the first configuration.

Aspect 32 can include, or can optionally be combined with the subject matter of Aspect 31, to optionally include wherein in the first configuration, an apex of the anterior side and an apex of the posterior side each projects a similar distance away from the longitudinal axis.

Aspect 33 can include, or can optionally be combined with the subject matter of Aspect 31 or Aspect 32, to optionally include wherein in the implanted configuration, the anterior side projects approximately 10 percent to approximately 40 percent further from the longitudinal axis than in the first configuration.

Aspect 34 can include, or can optionally be combined with the subject matter of any of Aspects 31-33, to optionally include the injector nozzle has a nozzle end between the nozzle cavity and the nozzle end opening; the nozzle end has a smaller diameter than the nozzle cavity; and the nozzle end opening has an angular profile.

Aspect 35 can include an implant insertion system comprising: a breast implant; and a marking tool including: a center point corresponding to a patient's nipple; at least one dimensional indicator configured to identify a target site for the breast implant; and a guiding line configured to locate a passage extending from an incision to the target site.

Aspect 36 can include, or can optionally be combined with the subject matter of Aspect 35, to optionally include the marking tool includes a plurality of dimensional indicators corresponding to breast implants of different sizes.

Aspect 37 can include, or can optionally be combined with the subject matter of any of Aspect 35 or Aspect 36, to optionally include the marking tool further includes an incision location identifier corresponding to a location of the incision into the passage.

Aspect 38 can include, or can optionally be combined with the subject matter of any of Aspects 35-37, to optionally include the marking tool identifies a final dissection mark at a junction between the at least one dimensional indicator and the guiding line.

Aspect 39 can include, or can optionally be combined with the subject matter of any of Aspects 35-38, to optionally include a tissue dissector configured for insertion to the final dissection mark.

Aspect 40 can include, or can optionally be combined with the subject matter of any of Aspects 35-39, to optionally include a tissue expander configured for insertion to the final dissection mark.

Aspect 41 can include, or can optionally be combined with the subject matter of any of Aspects 35-40, to optionally include the tissue expander has a compressed configuration and an expanded configuration, and wherein in the compressed configuration, the tissue expander is sized to be inserted through the incision.

Aspect 42 can include, or can optionally be combined with the subject matter of any of Aspects 35-41, to optionally include the marking tool having an incision location identifier configured to identify a length of the incision; a tissue dissector; a tissue expander; and an injector which houses the breast implant and insert the breast implant into the target site through the incision. The at least one of the tissue dissector, the tissue expander and injector have an insertion dimension substantially similar to the length of the incision or smaller than the length of the incision.

Aspect 43 can include, a method of locating an implant within a target site of a patient's body comprising: placing a marking tool on a breast of a patient with a center of the marking tool substantially aligned with a nipple; marking a surgical area using a plurality of reference lines on the breast including: tracing along at least one of the plurality of a reference lines corresponding to a dimensional indicator; and tracing along a navigation line corresponding to a passage extending from an incision location to a target site.

Aspect 44 can include, or can optionally be combined with the subject matter of Aspect 43, to optionally include the reference marking indicates a circumferential outline of the implant.

Aspect 45 can include, or can optionally be combined with the subject matter of any of Aspect 43 or Aspect 44, to optionally include the marking tool has at least two dimensional indicators corresponding to different sizes of implants.

Aspect 46 can include, or can optionally be combined with the subject matter of any of Aspect 43-45, to optionally include marking a final dissection mark corresponding to a final distance of insertion of a tissue dissector.

Aspect 47 can include, or can optionally be combined with the subject matter of any of Aspect 43-46, to optionally include marking an incision location identifier having lateral ends; and marking parallel lines between each of the lateral ends and the dimensional indicator.

Aspect 48 can include, or can optionally be combined with the subject matter of any of Aspect 43-47, to optionally include the incision location identifier is between approximately 1.0 centimeters and approximately 3.0 centimeters.

Aspect 49 can include, or can optionally be combined with the subject matter of any of Aspect 43-48, to optionally include the incision location identifier corresponds an incision to be made along an axillary skin crease.

Each of these non-limiting aspects can stand on its own or can be combined in various permutations or combinations with one or more of the other aspects.

The above description includes references to the accompanying drawings, which form a part of the detailed description. The drawings show, by way of illustration, specific examples in which the invention can be practiced. These examples are also referred to herein as “aspects” or “embodiments.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) can be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as aspects, examples or embodiments, with each claim standing on its own as a separate example, and it is contemplated that such examples can be combined with each other in various combinations or permutations. The scope of the invention should be determined with reference to the appended claims, along with the full scope of equivalents to which such claims are entitled.

The claimed invention is:

1. An implant insertion system comprising:
a tissue expander including an inflatable balloon, the balloon having a curved posterior side when inflated; and
an implant having a curved posterior side when positioned against an anatomical surface, wherein the curved posterior side of the implant substantially matches the curved posterior side of the tissue expander.

2. The implant insertion system of claim 1, wherein the implant further includes:
an anterior side;
a longitudinal axis positioned between the anterior side and the curved posterior side; and
a posterior apex disposed on the curved posterior side, the posterior apex extending further from the longitudinal axis in a first configuration than when the implant is positioned against the anatomical surface.

3. The implant insertion system of claim 2, wherein the anterior side of the implant is curved in a manner substantially similar to the curved posterior side, the anterior side of the implant includes an anterior apex, and the longitudinal axis is positioned substantially equidistant from the anterior apex and the posterior apex.

4. The implant insertion system of claim 1, wherein the implant further includes:
an anterior side having a curved profile with an apex substantially similar to a curved anterior side of the balloon.

5. The implant insertion system of claim 1, wherein the balloon has a compressed configuration and an inflated configuration.

6. The implant insertion system of claim 5, wherein when the balloon is in the compressed configuration the balloon is sized to fit through a small incision.

7. The implant insertion system of claim 6, wherein the balloon is configured to have a substantially similar diameter in the compressed configuration and the inflated configuration.

8. A method of forming a nest-like cavity within a breast area of a patient's body comprising:
making an incision having a specified dimension at a desired location of the patient's body;
inserting a tissue expander having a balloon into a target site of the patient's body;
inflating the balloon at the target site between at least two layers of connective tissue to separate the at least two layers of connective tissue and form a cavity with a profile substantially matching a profile of a breast implant;
wherein an anterior side of the balloon has a curved profile and a posterior side has a curved profile;
wherein as the balloon is inflated, the balloon is projected in an anterior direction and a posterior direction;
wherein when the balloon is substantially fully inflated, the at least two layers of connective tissue surround the balloon;
maintaining the balloon in an inflated state;
deflating the balloon after a predetermined period of time; and
removing the tissue expander and the balloon from the patient's body.

9. The method of forming the nest-like cavity within the breast area of the patient's body of claim 8, wherein the balloon is inflated to expand anteriorly to separate the connective tissue.

10. The method of forming the nest-like cavity within the breast area of the patient's body of claim 9, wherein the at least two layers of connective tissue are at least one of ligaments and soft tissue;
wherein when the at least one of ligaments and soft tissue are minimally separated around the posterior side of the balloon.

11. The method of forming the nest-like cavity within the breast area of the patient's body of claim 8, wherein the at least two layers of ligaments surround, encase, hug or nest the balloon.

12. The method of forming the nest-like cavity within the breast area of the patient's body of claim 8, wherein after separation, the at least two layers of ligaments maintain a nearly original anatomical structure.

13. An implant insertion method comprising:
expanding a tissue expander to form a target site in a patient's body, the tissue expander having a curved posterior side that preserves at least one ligament point of attachment posteriorly of the tissue expander; and
inserting an implant into the target site in the patient's body, the implant having a curved posterior side supported by the at least one ligament point of attachment posteriorly of the implant.

14. The implant insertion method of claim 13, wherein the at least one ligament point of attachment is located:
laterally of a center point of the implant to resist lateral movement of the implant; or
inferiorly of the implant to resist inferior movement of the implant.

15. The implant insertion method of claim 13, wherein the tissue expander includes a balloon having a compressed configuration and an expanded configuration.

16. The implant insertion method of claim 13, further comprising:
expanding the tissue expander to separate at least two layers of connective tissue at the target site to form a cavity while maintaining the at least one ligament point of attachment posteriorly of the tissue expander;
wherein the tissue expander comprises a balloon that projects in a posterior direction and in an anterior direction to separate the at least two layers of ligaments.

17. The implant insertion method of claim 16, wherein the at least two layers of connective tissue further includes ligaments;
wherein the ligaments wrap around and support the implant when the implant is inserted into the target site.

18. The implant insertion method of claim 13, further comprising:
making a small incision in a desired area of the patient's body;
inserting the tissue expander through the small incision; and
inserting the implant through the small incision.

19. The implant insertion method of claim 18, wherein the implant is inserted into the target site with an injector.

20. The implant insertion method of claim 13, wherein the target site is in a patient's breast area.

21. The implant insertion method of claim 13, wherein the tissue expander, when in an expanded state, is sized and shaped similar to the implant.

22. The implant insertion method of claim 13, wherein inserting the implant into the target site comprises:
loading the implant into an injector having a nozzle with a cavity, a nozzle end and a nozzle end opening, wherein the implant is compressed within the cavity of the nozzle between the nozzle end and an expandable membrane;
engaging an actuator on the injector coupled with a pressurized fluid source and coupled with the expandable membrane;
wherein the fluid source provides pressurized fluid into the expandable membrane;
applying a force to the implant with the expandable membrane;
expelling the implant through the nozzle end and out of the nozzle end opening and into the target site; and
maintaining the at least one ligament point of attachment during and after expelling the implant into the target site.

23. An implant insertion system comprising:
an injector including a nozzle that defines a housing; and
an implant including an anterior side, a posterior side, and a longitudinal axis between the anterior side and the posterior side, the implant having:
a first configuration in which each of the anterior side and the posterior side projects away from the longitudinal axis, wherein the first configuration comprises a neutral configuration;
a second configuration in which each of the anterior side and the posterior side is compressed towards the longitudinal axis when contained within the housing of the nozzle; and
an implanted configuration in which the posterior side is compressed against an anatomical structure and the anterior side projects further from the longitudinal axis than when in the first configuration.

24. The implant insertion system of claim 23, wherein the anterior side and posterior side of the implant each projects a substantially similar distance away from the longitudinal axis in the first configuration.

25. The implant insertion system of claim 23, wherein the anterior side of the implant projects approximately 10 percent to approximately 40 percent further in the implanted configuration than in the first configuration.

26. The implant insertion system of claim 23, wherein the anterior side of the implant projects approximately 20 percent to approximately 30 percent further in the implanted configuration than in the first configuration.

27. The implant insertion system of claim 23, wherein the anterior side and the posterior side are substantially uniformly compressed in the second configuration.

28. The implant insertion system of claim 23, wherein the implant is a breast implant.

29. The implant insertion system of claim 23, wherein the implant is symmetrical about at least two axes.

30. The implant insertion system of claim 23, wherein the injector further includes:
a handle detachably coupled with the nozzle;
wherein the handle includes an expandable membrane configured to expand into the nozzle with a supply of a pressurized fluid;
wherein the handle includes an actuator to open and close a connection between a pressurized fluid source and the expandable membrane.

31. A method of inserting an implant having an anterior side, a posterior side, and a longitudinal axis between the anterior side and the posterior side into a patient's body comprising:
drawing the implant into a nozzle cavity of an injector nozzle to transform the implant from a first configuration in which both the anterior and posterior sides of the implant project away from the longitudinal axis to a second configuration in which both the anterior and posterior sides of the implant are compressed towards the longitudinal axis;
coupling a handle having an expandable membrane with the injector nozzle such that the implant is disposed between a nozzle end opening and the expandable membrane;
applying a force with the expandable membrane against a proximal end of the implant; and
expelling the implant through the nozzle end opening and into a target site;
wherein expelling the implant into the target site allows the implant to expand to an implanted configuration in which the posterior side is compressed against an anatomical structure and the anterior side projects further from the longitudinal axis than when in the first configuration.

32. The method of inserting an implant of claim 31, wherein in the first configuration, an apex of the anterior side and an apex of the posterior side each projects a similar distance away from the longitudinal axis.

33. The method of inserting an implant of claim 31, wherein in the implanted configuration, the anterior side projects approximately 10 percent to approximately 40 percent further from the longitudinal axis than in the first configuration.

34. The method of inserting an implant of claim 31, wherein:
the injector nozzle has a nozzle end between the nozzle cavity and the nozzle end opening;
the nozzle end has a smaller diameter than the nozzle cavity; and
the nozzle end opening has an angular profile.

35. An implant insertion system comprising:
a breast implant; and
a marking tool including:
a center point corresponding to a patient's nipple; at least one dimensional indicator configured to identify a target site for the breast implant; and
a guiding line configured to locate a passage extending from an incision to the target site.

36. The implant insertion system of claim 35, wherein the marking tool includes a plurality of dimensional indicators corresponding to breast implants of different sizes.

37. The implant insertion system of claim 35, wherein the marking tool further includes an incision location identifier corresponding to a location of the incision into the passage.

38. The implant insertion system of claim 35, wherein the marking tool identifies a final dissection mark at a junction between the at least one dimensional indicator and the guiding line.

39. The implant insertion system of claim 38, further comprising:
a tissue dissector configured for insertion to the final dissection mark.

40. The implant insertion system of claim 38, further comprising:
a tissue expander configured for insertion to the final dissection mark.

41. The implant insertion system of claim 40, wherein the tissue expander has a compressed configuration and an expanded configuration, and wherein in the compressed configuration, the tissue expander is sized to be inserted through the incision.

42. The implant insertion system of claim 35 further comprising:
the marking tool having an incision location identifier configured to identify a length of the incision;
a tissue dissector;
a tissue expander; and
an injector which houses the breast implant and insert the breast implant into the target site through the incision;
wherein at least one of the tissue dissector, the tissue expander and injector have an insertion dimension substantially similar to the length of the incision or smaller than the length of the incision.

43. A method of locating an implant within a target site of a patient's body comprising:
placing a marking tool on a breast of a patient with a center of the marking tool substantially aligned with a nipple; and
marking a surgical area using a plurality of reference lines on the breast including:
tracing along at least one of the plurality of a reference lines corresponding to a dimensional indicator; and
tracing along a navigation line corresponding to a passage extending from an incision location to a target site.

44. The method of locating the implant of claim 43, wherein the reference marking indicates a circumferential outline of the implant.

45. The method of locating the implant of claim 43, wherein the marking tool has at least two dimensional indicators corresponding to different sizes of implants.

46. The method of locating the implant of claim 43, further comprising:
marking a final dissection mark corresponding to a final distance of insertion of a tissue dissector.

47. The method of locating the implant of claim 43, further comprising:
marking an incision location identifier having lateral ends; and
marking parallel lines between each of the lateral ends and the dimensional indicator.

48. The method of locating the implant of claim 47, wherein the incision location identifier is between approximately 1.0 centimeters and approximately 3.0 centimeters.

49. The method of locating the implant of claim 47, wherein the incision location identifier corresponds an incision to be made along an axillary skin crease.
